(19) United States
(12) Patent Application Publication
(10) Pub. No.: US 2025/0306038 A1
(43) Pub. Date: Oct. 2, 2025

(54) BLOOD BIOMARKERS OF OLIGODENDROCYTE-DERIVED EXOSOMES AND THEIR USE IN IDENTIFYING ASYMPTOMATIC BRAIN INJURY

(71) Applicant: Nanosomix, inc., Aliso Viejo, CA (US)

(72) Inventor: Masato Mitsuhashi, Irvine, CA (US)

(73) Assignee: Nanosomix, inc., Aliso Viejo, CA (US)

(21) Appl. No.: 19/097,521

(22) Filed: Apr. 1, 2025

Related U.S. Application Data
(60) Provisional application No. 63/572,450, filed on Apr. 1, 2024.

(51) Int. Cl. G01N 33/68

(52) U.S. Cl. CPC G01N 33/6896; G01N 2800/28; G01N 2800/52

(57) ABSTRACT

Methods of identifying and assays designed to identify at least one biomolecule from a patient are disclosed that comprise: collecting at least one biofluid from a patient, isolating at least one exosome from the biofluid, and identifying at least one biomolecule from the at least one exosome, wherein the at least one biomolecule is bound to the at least one exosome and is locally released from the patient. In some embodiments, the at least one biomolecule comprises a secretory protein, a neurotrophic factor, a growth factor, a cytokine, a chemokine, a pre-toxic molecule, a toxic molecule, or a combination thereof.

This application is a United States Utility Patent Application that claims priority to U.S. Provisional Patent Application Ser. No. 63/572,450 entitled “Assessment of Asymptomatic Acute Brain Injuries by Blood Biomarkers of Oligodendrocyte-derived Extracellular Vesicles” that was filed on Apr. 1, 2024, which is commonly owned and incorporated herein by reference in its entirety.

### FIELD OF THE SUBJECT MATTER

The field of the subject matter is blood biomarkers of oligodendrocyte-derived exosomes and their use in identifying asymptomatic brain injury, including subconcussive brain injury.

### BACKGROUND

Head trauma occurs after sports injuries, falls, motor vehicle accidents, assaults, abuse, workplace accidents, military activities, etc. and induces profound effects on physical, cognitive, and emotional health. According to the World Health Organization (WHO) (1), traumatic brain injuries (TBI) are a major cause of death and disability worldwide, particularly among young people, those in low- and middle-income countries, and people living in conflict or war-like zone.

A concussion is a mild head injury typically resolving within a few days. However, some patients experience prolonged problems lasting from weeks to months, known as post-concussion syndrome (PCS) (2). Even after symptoms have disappeared, we do not know whether the brain has fully returned to its pre-injury state. This uncertainty arises from the intricate nature of the brain, where subtle local damage may remain undetected as the brain employs alternate pathways to compensate. Hence, the absence of symptoms or the presence of seemingly innocuous ones does not necessarily signify the brain's complete recovery. These lingering issues may potentially contribute to futur0e complications such as learning disabilities, post-traumatic stress disorder (PTSD), chronic traumatic encephalopathy (CTE), suicide, etc.

Severe TBI often leads to complications like bleeding, infarction, and brain edema, which can be identified through advanced imaging techniques such as computed tomography (CT) or magnetic resonance imaging (MRI). Additionally, blood tests measuring neuronal and glial proteins are available for diagnostic purposes (3). However, these tests cannot always detect minor cellular and molecular damage. Physical examinations, such as eye movement, hearing, balance, and memory, etc. are dependent on the damage of respective sensory/motor pathways. Standardized Assessment of Concussion (SAC) (4), Sports Concussion Assessment Tool (SCAT) (5), Immediate Post-concussion Assessment (ImPACT) (6), etc. were established for the management of concussion athletes for the decision of return-to-play. However, the lack of objective and quantitative methods limits the conclusive nature of such diagnoses and subsequent management.

Due to the pioneering work by Dr. Omalu (7-8), which became a movie “Concussion” in 2015, and the death of famous boxer, Muhammed Ali in 2016, and American football player, Aaron Hernandez in 2017, long term consequences of concussion became a major public issue. Recently, concussion has been shown to no longer be a predominant male issue, and females have shown an equal or even higher incidence of concussion than males (9). The word “subconcussion” or “subconcussive condition” appeared in the title of scientific papers in 2009 (10), then appeared in a couple papers in each year thereafter. Although these papers alarmed the public for the risk of a subconcussive condition, we have limited resources for the assessment of asymptomatic subconcussive condition.

According to the Centers for Disease Control and Prevention (CDC), subconcussion refers to a brain condition after sudden head movement (e.g. in a hard tackle) without neurological symptoms (11). The term subconcussion is inherently ambiguous, as it encompasses both benign conditions and more serious subclinical traumatic brain injuries (TBI) (12). Given the brain's complexity as a highly interconnected network, minor injuries often go unnoticed, with the brain compensating for damage by rerouting functions through alternative neural pathways as shown and summarized in the legend in FIG. 1A. Consequently, the absence of symptoms immediately following a single event, days or weeks after a concussion, or from repetitive mild head impacts does not guarantee that the brain is unharmed.

Speaking of FIG. 1, these Figures show A. Asymptomatic condition: The brain operates as a highly complex network, and minor injuries may go unnoticed as the brain compensates by rerouting functions through alternative pathways, potentially leaving the individual asymptomatic. B. Advantage of exosomes: Current blood-based biomarkers typically focus on intracellular proteins released from dying or dead brain cells, with levels peaking at the time of injury and gradually declining (top). In contrast, exosomes are released from healthy, stressed, or activated brain cells-not from dead cells. Consequently, the levels of exosomes can fluctuate during the repair process, reflecting the dynamic nature of cellular activity following injury. C. Assay principle: When the myelin sheath is damaged-whether through compression, stretching, cracking, or twisting-various biomolecules (such as neurotrophic factors and inflammatory cytokines) are released at the injury site, where oligodendrocyte-derived exosomes are present. These biomolecules bind to the surface of exosomes and are then released into the bloodstream. By measuring these biomolecule-bound exosomes in peripheral blood, we can potentially monitor the cascade of cellular and molecular events following axonal injury.

In cases of either benign subconcussion or subclinical TBI, imaging techniques such as computed tomography (CT) and magnetic resonance imaging (MRI) typically fail to detect abnormalities. Moreover, athletes often forgo these costly imaging tests due to their lack of symptoms. Current blood-based diagnostic tests (i-STAT TBI, Abbott, Abbott Park, IL) focus on detecting proteins like ubiquitin C-terminal hydrolase L1 (UCHL1) and glial fibrillary acidic protein (GFAP) (13). However, because these are intracellular proteins from neurons and astrocytes respectively, these biomarkers lack the sensitivity required to identify subtle but potentially critical asymptomatic subconcussive conditions (FIG. 1B). Additionally, their concentrations peak shortly after the injury and then gradually decline, making them unsuitable for monitoring subsequent cellular recovery processes or neuroinflammatory cascade (FIG. 1B). Despite growing public awareness of the potential association of subconcussion with other conditions (14)—including depression, insomnia, learning disabilities, memory loss, personality changes, suicidal thoughts, chronic traumatic encephalopathy (CTE), and Parkinson's disease (PD)—subconcussion remains a conceptual medical term without clear, practical diagnostic criteria.

Numerous studies on concussion have mentioned subconcussion or subconcussive conditions. However, many of these studies primarily focus on symptomatic patients who seek care in clinics, emergency rooms, or hospitals. As a result, asymptomatic individuals with subconcussive injuries, who do not visit medical facilities, remain largely understudied. In this study, we sought to differentiate subclinical TBI from benign subconcussion by utilizing novel biomarkers and innovative clinical research models.

This persistent challenge of properly identifying and addressing asymptomatic brain injury is tackled by a groundbreaking concept by showing a new blood test format, which has the potential to revolutionize the landscape of concussion and TBI-related management and prevention.

To this end, it would be desirable to: a) utilize a blood test to identify and/or diagnose asymptomatic brain trauma or injury; b) develop and utilize locally released biomolecules to detect or diagnose asymptomatic brain trauma or injury, such as a subconcussion; c) to develop and utilize locally released biomolecules to detect or diagnose asymptomatic brain trauma or injury using as little as 5 μL of plasma from a patient; d) to develop and utilize locally released biomolecules to detect or diagnose asymptomatic brain trauma or injury that is noninvasive; and e) to assess post-trauma-event cellular cascades by monitoring the quantitative changes of BDNF, NRG1, and CNTF on the surface of ODE by utilizing a simple and straightforward blood test.

### SUMMARY OF THE SUBJECT MATTER

Methods of identifying at least one biomolecule from a patient are disclosed that comprise: collecting at least one biofluid from a patient, isolating at least one exosome from the biofluid, and identifying at least one biomolecule from the at least one exosome, wherein the at least one biomolecule is bound to the at least one exosome and is locally released from the patient. In some embodiments, the at least one biomolecule comprises a secretory protein, a neurotrophic factor, a growth factor, a cytokine, a chemokine, a pre-toxic molecule, a toxic molecule, or a combination thereof.

Assays are contemplated herein, wherein the assay comprises the steps of: obtaining a biological sample comprising at least one vesicle from a patient, enriching the at least one vesicle, such that the at least one vesicle expresses a first biomarker and a second biomarker.

### BRIEF DESCRIPTION OF THE FIGURES

FIG. 1: Rationale of the study. A. Asymptomatic condition. The brain operates as a highly complex network, and minor injuries may go unnoticed as the brain compensates by rerouting functions through alternative pathways, potentially leaving the individual asymptomatic. B. Advantage of exosomes. Current blood-based biomarkers typically focus on intracellular proteins released from dying or dead brain cells, with levels peaking at the time of injury and gradually declining (top). In contrast, exosomes are released from healthy, stressed, or activated brain cells—not from dead cells. Consequently, the levels of exosomes can fluctuate during the repair process, reflecting the dynamic nature of cellular activity following injury. C. Assay principle. When the myelin sheath is damaged—whether through compression, stretching, cracking, or twisting—various biomolecules (such as neurotrophic factors and inflammatory cytokines) are released at the injury site, where oligodendrocyte-derived exosomes are present. These biomolecules bind to the surface of exosomes and are then released into the bloodstream. By measuring these biomolecule-bound exosomes in peripheral blood, we can potentially monitor the cascade of cellular and molecular events following axonal injury.

FIG. 2: Cellular and biochemical cascade of post-TBI event. A: Healthy state. A neuron consists of a cell body and a long arm known as an axon, which extends toward synaptic end. Axons are insulated by myelin sheath produced and managed by oligodendrocytes (ODC). ODC release extracellular vesicles (EV), and ODC-derived EV (ODE) accumulate around axonal region. B: After mild injury. The pliability of neuronal cell bodies enables them to withstand mild mechanical forces, whereas axons, shielded by the protective myelin sheath, exhibit greater rigidity, rendering them more vulnerable to compression, stretch, squeeze, crack, etc. Then, neurons release 3 primary factors, such as brain-derived neurotrophic factor (BDNF), neureglin-1 (NRG1), and ciliary neurotrophic factor (CNTF). These factors migrate to damaged axonal lesions, and bind to respective receptors (TrkB for BDNF, ErbB for NRG1, and CNTFR for CNTF) expressed on the surface of ODC. These factors also bind to the receptors on ODE as well as non-specifically binding to the surface of ODE. These ODE are eventually entering the bloodstream.

FIG. 3: Preliminary data of soccer heading practice. Views A1 to D1: Plasma levels of CD9⁺MOG⁺ (A1), BDNF⁺MOG⁺ (B1), CNTF⁺MOG⁺ (C1), and NRG1⁺MOG⁺ (D1) as measured in an enzyme-linked immunosorbent assay (ELISA) with readings expressed as relative light units (RLU). Plasma samples were from 17 subjects before heading practice. Arrows: values of background (BKG) without plasma sample. Dotted lines: cutoff. Views A2 to D2: % from the baseline (before heading practice) of CD9⁺MOG⁺ (A2), BDNF⁺MOG⁺ (B2), CNTF⁺MOG⁺ (C2), and NRG1⁺MOG⁺ (D2), respectively. Views B3 to D3: B2-D2 data were normalized by the fluctuation of the amounts of ODE (A2).

FIG. 4: Validation of capture antibody. A. MOG-specificity. Seven different control plasma samples (•) and phosphate-buffered saline (PBS) (∘) were applied to ELISA wells coated with anti-MOG (Y-axis) and control rabbit IgG (X-axis). After washing away unbound materials, the wells were exposed to biotinylated anti-CD9 probes to quantify the captured CD9⁺ exosomes. The assay details are described in the Methods section. B. MOG reduction. Seven control plasma samples and a PBS sample were applied to the first anti-MOG-coated ELISA wells to capture MOG⁺ exosomes. The supernatants were then transferred to a second anti-MOG-coated well to capture any remaining MOG⁺ exosomes. Both wells were probed with anti-CD9 to quantify the amounts of captured CD9⁺ exosomes. To normalize the data, plasma values were subtracted by PBS values. The Y-axis represents the results from the first wells (pre-reduction), and the X-axis represents the results from the second wells (post-reduction). C. Nanoparticle tracking analysis (NTA). Captured exosomes on anti-MOG-immobilized paramagnetic beads were eluted and analyzed using NTA to confirm the presence and size distribution of exosomes. Assay details are described in the Methods section. The X-axis represents the particle size, and the Y-axis represents the number of particles detected at each size. D. Enrichment of ODEs. Control plasma and buffer (PBS) were applied to mouse IgG (mIgG) and anti-CD81 (exosome marker, mouse IgG) immobilized ELISA wells for the capture of whole exosomes, as well as rabbit IgG (rIgG) and anti-MOG (rabbit IgG) immobilized ELISA wells for the capture of ODEs. The first set of wells was probed with anti-CD81 to quantify the captured exosomes (D), and the second set was probed with anti-TRKB to quantify ODEs (E). The amounts of CD81⁺ whole exosomes captured in anti-MOG wells were 42% of those captured in anti-CD81 wells (D), whereas the amounts of TRKB⁺ ODE in anti-MOG wells were 1,162% of those captured in anti-CD81 wells (E).

FIG. 5: Validation of probes. A. Standard plasma dilution with various control plasma samples. Left. Our standard plasma was assigned a concentration of 100 units/mL, and dilution studies were conducted for IL1B (top), CNTF (middle), and SNCA (bottom). The Y-axis represents Relative Light Units (RLU) on a log scale. On the right, the results of 16 (IL1B and CNTF) and 26 (SNCA) different control plasma samples are shown, measured on the same scale as the standard plasma dilution. B. MOG reduction. The quantity of MOG⁺ ODE was reduced as shown in FIG. 2B. The Y-axis shows RLU before MOG reduction, and the X-axis shows RLU after MOG reduction. C. Quantification. Various volumes of plasma (0, 2.5, 5, 10, and 15 μL) from high (left) and low titer (right) plasma samples were applied to anti-MOG wells in triplicate and probed with anti-IL1B (top), anti-CNTF (middle), and anti-SNCA (bottom), respectively. * Indicates p<0.05 by unpaired t-test.

FIG. 6: Results of [CNTF on MOG]. A-C: Boxers and MMA fighters (n=18 in A, n=16 in B-C (2 were under detection limits). D: American football players (n=12). E: Cross-country athletes (n=4). A: [CD9 on MOG]. B: [IL1B on MOG]. C-E: [CNTF on MOG]. Symbols (∘) and bars were mean±standard deviation of whole subject (A, B, D, E) or stable subjects only (n=14, C). 2 individual data were shown in C•. X axis in A-C: before and after the bout. D-E: Blood collection once a month from July to November. Y axis: % from baseline (1^{st} blood collection in each subject).

FIG. 7: Results of [IL1B on MOG]—High school cross-country and American football. A: High school cross-country athletes. B-G: American football players from 6 different high schools (n=10, 47, 23, 13, 20, and 24 in each school, total 137 subjects, 919 plasma samples). X axis: Blood collection once a month from July to November (A-G except E), and before each game and pre-July) and post-season (November) in E. Y axis: [IL1B on MOG], % from baseline (1^{st} blood collection in each subject). Symbols (∘) and bars are mean±standard deviation of whole subject (A) or stable subjects only (B-E). Subjects with >200% are shown individually in B-E•.

FIG. 8: Results of [IL1B on MOG]—College rugby. A: Rugby players who did not show >200% (n=10). Since start date is not uniform, each subject data is shown. Nine were no concussion and 1 showed concussion twice (solid line). B. Case #1-3. Case #1 and 3 showed no concussion, and #2 showed concussion 4 times as indicated by solid triangles. C. Case #4 with different y-axis scale. Y axis: [IL1B on MOG], % from baseline (1^{st} blood collection in each subject).

FIG. 9: Results of [SNCA on MOG]. A: Boxers and MMA fighters. Symbols (∘) and bars are mean±standard deviation of all subjects (n=10). B. Rugby players who showed stable [SNCA on MOG] values (n=8). Subject #3 (•), who showed an increase in [IL1B on MOG] in FIG. 8, was included in this group. C. Rugby players who showed a transient >150% increase in [SNCA on MOG]. Subject #1 and #4 showed an increase in [IL1B on MOG] (as shown in FIG. 8), while subject #5 did not. None of the players showed concussion symptoms. D. Rugby players who showed a continuous upward trend of [SNCA on MOG] (subject #2 and #6) and no increase despite two concussion episodes (subject #7). Subject #2 experienced 4 concussions and showed an increase in [IL1B on MOG] (FIG. 8). Subject #6 had no concussions and showed no increase in [IL1B on MOG]. The time of concussion is shown in open and solid triangles.

### DETAILED DESCRIPTION

As disclosed and discussed herein, contemplated methods, compositions, and tests: a) utilize a blood test to identify and/or diagnose asymptomatic brain trauma or injury; b) utilize locally released biomolecules to detect or diagnose asymptomatic brain trauma or injury, such as a subconcussion; c) utilize locally released biomolecules to detect or diagnose asymptomatic brain trauma or injury using as little as 5 μL of plasma from a patient; d) utilize locally released biomolecules to detect or diagnose asymptomatic brain trauma or injury that is noninvasive; and e) assess post-trauma-event cellular cascades by monitoring the quantitative changes of BDNF, NRG1, and CNTF on the surface of ODE by utilizing a simple and straightforward blood test.

It is to be understood that contemplated embodiments are not limited to the particular methodologies, protocols, cell lines, assays, and reagents described herein, as these may vary. It is also to be understood that the terminology used herein is intended to describe particular embodiments and is in no way intended to limit the scope of contemplated embodiments as set forth in the appended claims.

It must be noted that as used herein and in the appended claims, the singular forms “a,” “an,” and “the” include plural references unless context clearly dictates otherwise. Thus, for example, a reference to “a fragment” includes a plurality of such fragments, a reference to an “antibody” is a reference to one or more antibodies and to equivalents thereof known to those skilled in the art, and so forth.

Unless defined otherwise, all technical and scientific terms used herein have the same meanings as commonly understood by one of ordinary skill in the art to which these contemplated embodiments and disclosure belongs. Although any methods and materials similar or equivalent to those described herein can be used in the practice or testing of contemplated embodiments, the preferred methods, devices, and materials are now described. All publications cited herein are incorporated herein by reference in their entirety for the purpose of describing and disclosing the methodologies, reagents, and tools reported in the publications that might be used in connection with contemplated embodiments. Nothing herein is to be construed as an admission that contemplated embodiments and elements of the present disclosure are not entitled to antedate such disclosure by virtue of prior invention.

The practice of the embodiments and contemplated embodiments of the disclosure will utilize, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

Methods of identifying at least one biomolecule from a patient are disclosed that comprise: collecting at least one biofluid from a patient, isolating at least one exosome from the biofluid, and identifying at least one biomolecule from the at least one exosome, wherein the at least one biomolecule is bound to the at least one exosome and is locally-released from the patient. In some embodiments, the at least one biomolecule comprises a secretory protein, a neurotrophic factor, a growth factor, a cytokine, a chemokine, a pre-toxic molecule, a toxic molecule, or a combination thereof. As used herein, the phrase “toxic molecule” is used to refer to physiological and pathological forms of alpha-synuclein, amyloid beta, and tau proteins. Contemplated pathological forms include monomeric, polymeric, aggregated, phosphorylated, acetylated, or glycosylated.

It should be understood that the terms “exosome” and “EV” are used interchangeably herein. Exosomes range in size from 30 to 150 nanometers, are membrane-bound extracellular vesicles (EVs) that are produced in the endosomal compartment of most eukaryotic cells. In multicellular organisms, exosomes and other EVs are found in biological fluids including saliva, blood, urine and cerebrospinal fluid. EVs have specialized functions in physiological processes, from coagulation and waste management to intercellular communication.

In some contemplated embodiments, the at least one biomolecule comprises a secretory protein, a neurotrophic factor, a growth factor, a cytokine, a chemokine, a pre-toxic molecule, a toxic molecule, or a combination thereof. In contemplated embodiments, the at least one biomolecule is used to identify asymptomatic brain injury or trauma in the patient. In other contemplated embodiments, the at least one biomolecule is used to identify a severity of asymptomatic brain injury or trauma in the patient. And in yet other contemplated embodiments, the at least one biomolecule is used to identify the success of treatment of asymptomatic brain injury or trauma in the patient as compared with an initial level of the at least one biomolecule that was used to identify a severity of asymptomatic brain injury or trauma in the patient.

In some contemplated embodiments, the at least one biofluid is selected from blood, cerebrospinal fluid, urine, saliva, stool, luminal fluid, ascites, pleural effusion, or a combination thereof. In other contemplated embodiments, the at least one biofluid comprises at least two independent exosomes, wherein the at least two independent exosomes are different from one another.

In some contemplated embodiments, the at least one exosome is neuron-derived, astrocyte-derived, oligodendrocyte-derived, tumor-derived, microglia-derived, or a combination thereof. In other contemplated embodiments, the at least one oligodendrocyte-derived exosome is isolated by anti-MOG.

Assays are contemplated herein, wherein the assay comprises the steps of: obtaining a biological sample comprising at least one vesicle from a patient, enriching the at least one vesicle, such that the at least one vesicle expresses a first biomarker and a second biomarker. In some contemplated embodiments, the assay is used to identify asymptomatic brain injury or trauma in the patient.

Some contemplated assay embodiments further comprise measuring the level of the at least one vesicle in the biological sample, and comparing the level of the at least one vesicle in the biological sample to a control level of the at least one vesicle in a control biological sample, wherein an increase in the at least one vesicle in the biological sample as compared to the control level of the at least one vesicle in a control biological sample is an indicator of asymptomatic brain injury or trauma in the patient.

In yet other contemplated embodiments, the control biological sample comprises a biological sample from the same patient at a different and/or future point in time. As discussed herein, an athlete may have a sample taken from him or her before he or she starts participating in a sports program. This same would be considered a control biological sample- or one that can be used as a baseline comparison sample at a future point in time. These control biological samples could be saved or stored by the institution or team to be used at a future date to compare with a future biological sample from the same athlete. This future point in time could be after a concussive experience or at a non-activity-based future event, such as the end of the sports season. The reason for comparing a control biological sample to a biological sample at a non-activity-based future event may be to see if the athlete has developed any asymptomatic brain injury or trauma during the season from just every day wear and tear during the sports activity.

In some contemplated assay embodiments, the at least one vesicle comprises an extracellular vesicle that is derived from at least one oligodendrocyte. In yet other embodiments, the first biomarker comprises myelin oligodendrocyte glycoprotein.

In some contemplated embodiments, the second biomarker comprises a neuron-specific protein (e.g., synaptosome associated protein 25 (SNAP25), neurogranin (NRGN), tau, phosphorylated tau, αβ-42, αβ-40, along with aggregated forms, and synaptophysin), an astrocyte-specific protein (e.g., glial fibrillary acidic protein (GFAP) and excitatory amino acid transporter 1 (EAAT1)), a microglia-specific protein (CD11b), an oligodendrocyte-specific protein (e.g., myelin basic protein (MBP), an oligodendrocyte myelin glycoprotein (OMG), a cytosolic protein (e.g., glyceraldehyde-3-phosphate dehydrogenase (GAPDH), alpha-synuclein (SNCA), cathepsin D (CTSD), AchE, LAMP1, REST, SYT, TH, SYP, SYNPO, PSD95, SV2A, GYS, HSP70, BACE, SYMPO, NEFL, caspase, ubiquitin, PSEN1, GSK, PLAP, CSH1, PSG1, or FasL), a chemokine (CX3CL1, CCLs, CXCLs) or cytokine (interleukins, such as IL1b, IL34, IL12B or FasL).

In some embodiments, a contemplated second biomarker comprises a neuron-specific protein (e.g., synaptosome associated protein 25 (SNAP25), neurogranin (NRGN), tau, phosphorylated tau, αβ-42, αβ-40, along with aggregated forms, and synaptophysin), an astrocyte-specific protein (e.g., glial fibrillary acidic protein (GFAP) and excitatory amino acid transporter 1 (EAAT1)), a microglia-specific protein (CD11b), an oligodendrocyte-specific protein (e.g., myelin basic protein (MBP), an oligodendrocyte myelin glycoprotein (OMG), a cytosolic protein (e.g., glyceraldehyde-3-phosphate dehydrogenase (GAPDH), alpha-synuclein (SNCA), cathepsin D (CTSD), AchE, LAMP1, REST, SYT, TH, SYP, SYNPO, PSD95, SV2A, GYS, HSP70, BACE, SYMPO, NEFL, caspase, ubiquitin, PSEN1, GSK, PLAP, CSH1, PSG1, or FasL), a chemokine (CX3CL1, CCLs, CXCLs) or cytokine (interleukins, such as IL1b, IL34, IL12B or FasL)

In some embodiments, a contemplated second biomarker comprises at least one member of the neurotrophin family; a nerve growth factor (NGF), which supports survival of sensory and sympathetic neurons; and a brain-derived neurotrophic factor (BDNF), which plays a critical role in synaptic plasticity, learning, and memory.

With respect to the neurotrophin family, contemplated embodiments include neurotrophin-3 (NT-3), which supports survival and differentiation of neurons in the peripheral and central nervous systems; neurotrophin-4/5 (NT-4/5), which is involved in neuronal survival and synaptic modulation; the glial cell line-derived neurotrophic factor (GDNF) family, which promotes survival of dopaminergic and motor neurons.

In other embodiments, a contemplated second biomarker comprises neurturin (NRTN) which supports parasympathetic and sensory neurons; artemin (ARTN), which promotes survival of sympathetic neurons; persephin (PSPN), which is involved in motor neuron survival; a member of the cytokine Family (Interleukin-6 Family), which plays a role in neuroinflammation and neuronal survival; ciliary Neurotrophic Factor (CNTF), which supports motor neurons and astrocyte function; leukemia Inhibitory Factor (LIF), which is involved in neuroprotection and development; fibroblast growth factor (FGF) family, including fibroblast growth factor-2 (FGF-2, bFGF), which is involved in neurogenesis, angiogenesis, and repair and fibroblast growth factor-8 (FGF-8), which plays a role in neural patterning; and other growth factors, including insulin-like growth factor-1 (IGF-1), which promotes neuronal survival and synaptic plasticity, platelet-derived growth factor (PDGF), which is involved in neuroprotection and oligodendrocyte development, vascular endothelial growth factor (VEGF), which supports neurogenesis and angiogenesis, transforming growth factor-β (TGF-β), which regulates neuronal differentiation and survival, and epidermal growth factor (EGF), which promotes neural stem cell proliferation.

Axons are considered as principal loci of interest after head trauma, due to the vulnerability of the myelin sheath to mechanical forces. After neurons detect such axonal problems, neurons release 3 primary factors, such as brain-derived neurotrophic factor (BDNF), ciliary neurotrophic factor (CNTF), and neureglin-1 (NRG1). These factors migrate to damaged axonal lesions, and bind to respective receptors (TrkB for BDNF, CNTFR for CNTF, and ErbB for NRG1) expressed on the surface of oligodendrocytes (ODC), thereby initiating the repair processes of the damaged myelin sheath. BDNF, CNTF, and NRG1 also bind to ODC-derived extracellular vesicles (ODE) exist around ODC, eventually entering the bloodstream. Therefore, by monitoring the quantitative changes of BDNF, CNTF, and NRG1 on plasma ODE, it may be possible to assess post-event cellular cascade by a simple blood test.

The blood test format introduced in this study was a sandwich immunoassay using anti-myelin oligodendrocyte glycoprotein (MOG) as an ODC capture agent and probes against BDNF, CNTF, and NRG1, respectively. As a preliminary trial using as small as 5 μL of plasma samples obtained from soccer heading practice, 2 athletes out of 17 showed a transient increase 24 hours after the practice and one returned to the baseline levels in 72 hours and the other still showed slightly high CNTF values at 72 hours. This approach offers a promising avenue for non-invasive evaluation of brain trauma, irrespective of symptomatic manifestation.

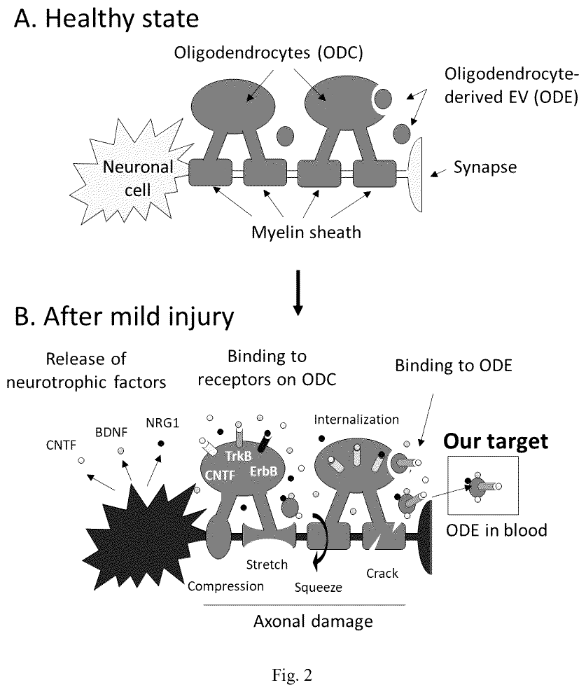

A neuron consists of a cell body and a long arm known as an axon, which extends toward synaptic end to transmit nerve impulses to other neuronal cells (FIG. 2A). Like electric wires, axons are insulated by myelin sheath produced and managed by oligodendrocytes (ODC) in the brain or Schwann cells in the periphery (FIG. 2A). ODC release extracellular vesicles (EV), and ODC-derived EV (ODE) accumulate around axonal region (FIG. 2A), eventually entering the bloodstream (15-17).

FIG. 2 shows cellular and biochemical cascade of post-TBI event. A: Healthy state. A neuron consists of a cell body and a long arm known as an axon, which extends toward synaptic end. Axons are insulated by myelin sheath produced and managed by oligodendrocytes (ODC). ODC release extracellular vesicles (EV), and ODC-derived EV (ODE) accumulate around axonal region. B: After mild injury. The pliability of neuronal cell bodies enables them to withstand mild mechanical forces, whereas axons, shielded by the protective myelin sheath, exhibit greater rigidity, rendering them more vulnerable to compression, stretch, squeeze, crack, etc. Then, neurons release 3 primary factors, such as brain-derived neurotrophic factor (BDNF), neureglin-1 (NRG1), and ciliary neurotrophic factor (CNTF). These factors migrate to damaged axonal lesions, and bind to respective receptors (TrkB for BDNF, ErbB for NRG1, and CNTFR for CNTF) expressed on the surface of ODC. These factors also bind to the receptors on ODE as well as non-specifically binding to the surface of ODE. These ODE are eventually entering the bloodstream.

### Biological Sample

A biological sample comprising vesicles (e.g., exosomes) may be obtained from a subject. The biological sample obtained from the subject is typically blood, but can be any sample from bodily fluids, tissue or cells comprising the vesicles to be analyzed. The biological sample may include, but is not limited to, whole blood, serum, plasma, urine, interstitial fluid, peritoneal fluid, cerebrospinal fluid, a cervical swab, tears, saliva, a buccal swab, skin, organs, and biopsies. Alternatively, exosomes can be obtained from cultured cells by collection of secreted exosomes from the surrounding culture media.

In some embodiments, the biological sample of the invention is obtained from blood. In some embodiments, about 1-10 mL of blood is drawn from a subject. In other embodiments, about 10-50 mL of blood is drawn from a subject. Blood can be drawn from any suitable area of the body, including an arm, a leg, or blood accessible through a central venous catheter. In some embodiments, blood is collected following a treatment or activity. For example, blood can be collected following a medical exam. The timing of collection can also be coordinated to increase the number and/or composition of vesicles (e.g., exosomes) present in the sample. For example, blood can be collected following exercise or a treatment that induces vascular dilation.

Blood may be combined with various components following collection to preserve or prepare samples for subsequent techniques. For example, in some embodiments, blood is treated with an anticoagulant, a cell fixative, a protease inhibitor, a phosphatase inhibitor, a protein, a DNA, or an RNA preservative following collection. In some embodiments, blood is collected via venipuncture using vacuum collection tubes containing an anticoagulant such as EDTA or heparin. Blood can also be collected using a heparin-coated syringe and hypodermic needle. Blood can also be combined with components that will be useful for cell culture. For example, in some embodiments, blood is combined with cell culture media or supplemented cell culture media (e.g., cytokines).

### Enrichment or Isolation of Vesicles (Exosomes, Microparticles, Microvesicles, Nanosomes, Extracellular Vesicles, and Ectosomes)

Samples can be enriched for vesicles through positive selection, negative selection, or a combination of positive and negative selection. In some embodiments, vesicles are directly captured. In other embodiments, blood cells are captured and vesicles are collected from the remaining biological samples. In some embodiments, the vesicles enriched in the biological samples are exosomes, microparticles, microvesicles, nanosomes, extracellular vesicles, or ectosomes. In some embodiments, the vesicles enriched in the biological samples are neuron-derived exosomes, astrocyte-derived exosomes, oligodendrocyte-derived exosomes, or microglia-derived exosomes.

Samples can also be enriched for vesicles based on differences in the biochemical properties of vesicles. For example, samples can be enriched for vesicles based on antigen, nucleic acid, metabolic, gene expression, or epigenetic differences. In some of the embodiments based on antigen differences, antibody-conjugated magnetic or paramagnetic beads in magnetic field gradients or fluorescently labeled antibodies with flow cytometry are used. In some of the embodiments based on nucleic acid differences, flow cytometry is used. In some of the embodiments based on metabolic differences, dye uptake/exclusion measured by flow cytometry or another sorting technology is used. In some of the embodiments based on gene expression, cell culture with cytokines is used. Samples can also be enriched for vesicles based on other biochemical properties known in the art. For example, samples can be enriched for vesicles based on pH or motility. Further, in some embodiments, more than one method is used to enrich for vesicles. In other embodiments, samples are enriched for vesicles using antibodies, ligands, or soluble receptors.

In other embodiments, surface markers are used to positively enrich vesicles in the sample. In some embodiments, the vesicles are exosomes, microparticles, microvesicles, nanosomes, extracellular vesicles, or ectosomes. In other embodiments, NCAM, CD171, CD9, CD63, CD81, SNAP25, EAAT1, OMG, MOG, neuron-specific enolase, diverse neuron or astrocyte adhesive proteins, microglial CD18/11, or CD3 T cell membrane cell surface markers are used to enrich for exosomes. In some embodiments, cell surface markers that are not found on vesicles populations are used to negatively enrich vesicles by depleting cell populations. Flow cytometry sorting may also be used to further enrich for exosomes using cell surface markers or intracellular or extracellular markers conjugated to fluorescent labels. Intracellular and extracellular markers may include nuclear stains or antibodies against intracellular or extracellular proteins preferentially expressed in vesicles. Cell surface markers may include antibodies against cell surface antigens that are preferentially expressed on exosomes (e.g., NCAM). In some embodiments, the cell surface marker is a neuron-derived exosome surface marker, including, for example, NCAM or CD171. In some embodiments, a monoclonal NCAM, CD9, CD63, CD81, neuron-specific enolase or CD171 antibody is used to enrich or isolate exosomes from the sample. In certain aspects, the NCAM, CD9, CD63, CD81, neuron-specific enolase or CD171 antibody is biotinylated. In this embodiment, biotinylated NCAM or CD171 antibody can form an antibody-exosome complex that can be subsequently isolated using streptavidin-agarose resin or beads. In other embodiments, the NCAM, CD9, CD63, CD81, neuron-specific enolase or CD171 antibody is a monoclonal anti-human NCAM, CD9, CD63, CD81, neuron-specific enolase or CD171 antibody. In other embodiments, the cell surface marker is a neuron-specific protein (e.g., synaptosome associated protein 25 (SNAP25), neurogranin (NRGN), tau, phosphorylated tau, αβ-42, and synaptophysin), an astrocyte-specific protein (e.g., glial fibrillary acidic protein (GFAP) and excitatory amino acid transporter 1 (EAAT1)), a microglia-specific protein (CD11b), an oligodendrocyte-specific protein (e.g., myelin basic protein (MBP), an oligodendrocyte myelin glycoprotein (OMG), a cytosolic protein (e.g., glyceraldehyde-3-phosphate dehydrogenase (GAPDH), alpha-synuclein (SNCA), cathepsin D (CTSD), AchE, LAMP1, REST, SYT, TH, SYP, SYNPO, PSD95, SV2A, GYS, HSP70, BACE, SYMPO, NEFL, caspase, ubiquitin, PSEN1, GSK, PLAP, CSH1, PSG1, or FasL), or a chemokine (CX3CL1) or cytokine (IL1b, IL34, FasL, or IL12B).

In some embodiments, enriched vesicles from the biological sample are subsequently enriched for a specific type of vesicle. For example, the biological sample is enriched for exosomes and then the enriched exosomes are subsequently enriched for neural-derived exosomes. In some embodiments, the biological sample is enriched for individual neural cell sources of vesicles. In certain aspects, the neural cell sources of vesicles are microglia, neurons, or astrocytes.

In other embodiments, vesicles are isolated or enriched from a biological sample by a method comprising: contacting a biological sample with an agent under conditions wherein a vesicle present in said biological sample binds to said agent to form a vesicle-agent complex; and isolating said vesicle from said vesicle-agent complex to obtain a sample containing said vesicle, wherein the purity of vesicles present in said sample is greater than the purity of vesicles present in said biological sample. In certain embodiments, the agent is an antibody or a lectin. Lectins useful for forming a vesicle-lectin complex are described in U.S. Patent Application Publication No. 2012/0077263.

In some embodiments, the vesicle is an exosome, a microparticle, a microvesicle, nanosomes, extracellular vesicles, or an ectosome. In some embodiments, the exosomes are neuron-derived exosomes, astrocyte-derived exosomes, oligodendrocyte-derived exosomes, or microglia-derived exosomes. In some embodiments, multiple isolating or enriching steps are performed. In certain aspects of the present embodiment, a first isolating step is performed to isolate exosomes from a blood sample and a second isolating step is performed to isolate neural-derived exosomes from other exosomes.

In yet other embodiments, the methods further comprise releasing the vesicle from the vesicle-agent complex. In other embodiments, the vesicle is released by exposing the vesicle-agent complex to low pH between 3.5 and 1.5. In other embodiments, the vesicle is released using a competing peptide that competes for the binding of the selection antibody used in the methods of the present invention. In yet other embodiments, the released vesicle is neutralized by adding a high pH solution. In other embodiments, the released vesicle is lysed by incubating the released vesicles with a lysis solution. In still other embodiments, the lysis solution contains inhibitors for proteases and phosphatases. It should be understood that there are contemplated embodiments where lysis of exosomes is not necessary or needed in order to carry out or implement contemplated embodiments of the present disclosure.

### Biomarkers

Biomarker levels on vesicles are assayed in a biological sample obtained from a subject having or at-risk of having a disease. In some embodiments, biomarker levels on vesicles are assayed in a biological sample obtained from a subject having or at-risk of having asymptomatic brain trauma. In some embodiments, one or more biomarkers are selected from the group consisting of a neuron-specific protein (e.g., synaptosome associated protein 25 (SNAP25), neurogranin (NRGN), tau, and synaptophysin), an astrocyte-specific protein (e.g., glial fibrillary acidic protein (GFAP) and excitatory amino acid transporter 1 (EAAT1)), a microglia-specific protein (CD11b), an oligodendrocyte-specific protein (e.g., myelin basic protein (MBP), an oligodendrocyte myelin glycoprotein (OMG)), and an extracellular vesicle-specific protein (dopamine transporter, DAT). In another embodiment, the biomarkers are CD171, phosphorylated tau T181, SNCA, and NRGN. In other embodiments, the biomarkers are acetylcholinesterase (AchE), Lysosomal Associated Membrane Protein 1 (LAMP1), CTSD, RE1 Silencing Transcription Factor (REST), synaptotagmin (SYT), monocyte chemotactic protein-1 (CCL2), IL34, glycogen synthase (GYS), (OR), death receptor 6 (DR6), heat shock protein (HSP), IL12beta, alpha-beta (AB), and beta-secretase (BACE). In some embodiments, one or more biomarkers are selected from the group consisting of cytosolic proteins, secretory proteins, membrane proteins and receptors and their pathological forms, including aggregates and mutated ones. Biomarkers of the present invention include neurotransmitter receptors, such as, for example, dopamine receptors (D1 and D2), serotonin receptors (2A, 2C, and 3B), GABA receptors (1-6, 5. B1, B2), and glutamate receptors (1 and 2). Other receptor biomarkers of the present invention include, insulin receptors, tumor necrosis factor receptors superfamily (TRAL, TNF receptor, death receptor 5 and 6), and neuropeptide receptors (orexin receptor, opioid receptor KOR). Biomarkers of the present invention include membrane proteins, such as, for example, EpCAM, PD-L1, ErbB2, CK19, TCR, CD16, CD28, CD32, CD79a, TREM2, and NCAM. Other known neurological disorder biomarkers may be used in combination with the biomarkers of the present invention. Examples of such biomarkers are provided in US Patent Application Pub. No. 2015/0119278, the contents of which are hereby incorporated by reference.

One of ordinary skill in the art has several methods and devices available for the detection and analysis of the markers of the instant disclosure. With regard to polypeptides or proteins on vesicles in patient test samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amounts of analytes without the need for a labeled molecule.

Preferably the markers are analyzed using an immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, an enzyme-linked immunosorbent assay (ELISA), immunofluorescent assay (IFA), immune-polymerase chain reaction assay, electro-chemiluminescence immunoassay (ECLIA), radioimmunoassay (RIA), competitive binding assay, planar waveguide technology, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the surface markers on vesicles is also contemplated by the present invention. The antibodies could be immobilized onto a variety of solid supports, such as magnetic or chromatographic matrix particles, the surface of an assay place (such as microtiter wells), pieces of a solid substrate material (such as plastic, nylon, paper), and the like. An assay strip could be prepared by coating the antibody or a plurality of antibodies in an array on solid support. This strip could then be dipped into the test sample to capture vesicles through binding to surface markers and then processed quickly through washes and detection steps with detection reagents, as described above, to generate a measurable signal, such as a colored spot.

The analysis of a plurality of markers may be carried out separately or simultaneously with one test sample. Several markers on vesicles may be captured and/or detected using a combination of multiple capture agents and/or detection agents in one test for efficient processing of multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease status that includes, but is not limited to identifying the approximate time from onset of the event, the presence and amount of salvageable tissue, the appropriateness of drug therapies, the effectiveness of various therapies, identification of the severity of the event, identification of the disease severity, and identification of the patient's outcome, including risk of future events.

An assay consisting of a combination of the markers referenced in the instant invention may be constructed to provide relevant information related to differential diagnosis. Such a panel may be constructed using 1, 2, 3, 4, 5, 6, 7, 8, 9, 10, 15, 20, or more or individual markers. The analysis of a single marker or subsets of markers comprising a larger panel of markers could be carried out methods described within the instant invention to optimize clinical sensitivity or specificity in various clinical settings.

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings. Particularly useful physical formats comprise surfaces having a plurality of discrete, addressable locations for the detection of a plurality of different analytes. Such formats include protein microarrays, or “protein chips” and capillary devices.

Biomarkers of the present disclosure serve an important role in the early detection and monitoring of asymptomatic brain trauma. Markers of such disorders are typically substances found in a bodily sample that can be measured. The measured amount can correlate to underlying disorder or disease pathophysiology, presence or absence of a neurological disorder, probability of a neurological disorder in the future. In patients receiving treatment for their condition the measured amount will also correlate with responsiveness to therapy. In some embodiments, a decrease or increase in the level of one or more biomarkers of the present invention is indicative of a neurological disorder. For example, an increase in phosphorylated tau T181 levels and/or a decrease in NRGN levels on exosomes having the CD171 membrane marker is indicative of Alzheimer's disease. Accordingly, the methods of the present invention are useful for the differential diagnosis of Alzheimer's disease.

In some embodiments, a biomarker is measured by a method selected from the group consisting of immunohistochemistry, immunocytochemistry, immunofluorescence, immunoprecipitation, electro-chemiluminescence immunoassay, radioimmunoassay, immune-polymerase chain reaction, western blotting, and ELISA.

### Clinical Assay Performance

The methods of the present invention may be used in clinical assays to diagnose or prognose asymptomatic brain trauma in a subject, identify a subject at risk of asymptomatic brain trauma, and/or for prescribing a therapeutic regimen or predicting benefit from therapy in a subject having asymptomatic brain trauma. Clinical assay performance can be assessed by determining the assay's sensitivity, specificity, area under the ROC curve (AUC), accuracy, positive predictive value (PPV), and negative predictive value (NPV). Disclosed herein are assays for diagnosing or prognosing asymptomatic brain trauma in a subject, identifying a subject at risk of asymptomatic brain trauma, or for prescribing a therapeutic regimen or predicting benefit from therapy in a subject having asymptomatic brain trauma.

The clinical performance of the assay may be based on sensitivity. The sensitivity of an assay of the present invention may be at least about 40%, 45%, 50%, 55%, 60%, 65%, 70%, 75%, 80%, 85%, 90%, 95%, 99%, or 100%. The clinical performance of the assay may be based on specificity. The specificity of an assay of the present invention may be at least about 40%, 45%, 50%, 55%, 60%, 65%, 70%, 75%, 80%, 85%, 90%, 95%, 99%, or 100%. The clinical performance of the assay may be based on area under the ROC curve (AUC). The AUC of an assay of the present invention may be at least about 0.5, 0.55, 0.6, 0.65, 0.7, 0.75, 0.8, 0.85, 0.9, or 0.95. The clinical performance of the assay may be based on accuracy. The accuracy of an assay of the present invention may be at least about 40%, 45%, 50%, 55%, 60%, 65%, 70%, 75%, 80%, 85%, 90%, 95%, 99%, or 100%.

### Compositions

Compositions useful in the methods of the present invention include compositions that specifically recognize a biomarker associated with asymptomatic brain trauma. Such compositions may include capture agents and/or detection agents that recognize, for example, a neuron-specific protein biomarker, such as synaptosome associated protein 25 (SNAP25), αβ-42, neurogranin (NRGN), tau, phosphorylated tau, and synaptophysin, an astrocyte-specific protein biomarker, such as glial fibrillary acidic protein (GFAP) and excitatory amino acid transporter 1 (EAAT1), an oligodendrocyte-specific protein biomarker, such as myelin basic protein (MBP) and oligodendrocyte myelin glycoprotein (OMG), a microglia-specific protein (CD11b), a cytosolic protein (e.g., glyceraldehyde-3-phosphate dehydrogenase (GAPDH), alpha-synuclein (SNCA), cathepsin D (CTSD), AchE, LAMP1, REST, SYT, TH, SYP, SYNPO, PSD95, SV2A, GYS, HSP70, BACE, SYMPO, NEFL, caspase, ubiquitin, PSEN1, GSK, PLAP, CSH1, PSG1, or FasL), or a chemokine (CX3CL1) or cytokine (IL1b, IL34, FasL, or IL12B).

In yet other embodiments, the composition is selected from the group consisting of a peptide, a nucleic acid, an antibody, and a small molecule.

In certain embodiments, the present invention relates to compositions that specifically detect a biomarker associated with asymptomatic brain trauma. As detailed elsewhere herein, the present invention is based upon the finding that GAPDH, CTSD, NRGN, MBP, GFAP, Tau, phosphorylated Tau, synaptophysin, αβ-42, CX3CL1, IL1b, IL34, CD81, CD63, CD171, SNAP25, EAAT1, SNCA, CD11b, OMG, AchE, LAMP1, REST, SYT, TH, SYP, SYNPO, PSD95, SV2A, CCL2, IL34, GYS, OR, DR6, HSP, IL12b, AB, and BACE can be used as biomarkers for AD and other neurological disorders. In some embodiments, the compositions of the present invention specifically bind to and detect such biomarkers. For example, a composition may comprise a solid support comprising capture agents associated therewith that selectively bind to CD81, CD63, CD171, SNAP25, EAAT1, CD11b, or OMG. In another example, a composition may comprise detection agents that selectively bind to GAPDH, CTSD, NRGN, MBP, GFAP, Tau, phosphorylated Tau, synaptophysin, αβ-42, SNCA, CX3CL1, IL1b, IL34, OMG, AchE, LAMP1, REST, SYT, TH, SYP, SYNPO, PSD95, SV2A, CCL2, IL34, GYS, OR, DR6, HSP, IL12b, AB, and/or BACE.

In some embodiments, the composition comprises an antibody, where the antibody specifically binds to a biomarker or vesicles of the invention. The term “antibody” as used herein and further discussed below is intended to include fragments thereof which are also specifically reactive with a biomarker or vesicle (e.g., exosome). Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. Antigen-binding portions may also be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), single domain antibodies, bispecific antibodies, chimeric antibodies, humanized antibodies, diabodies and polypeptides that contain at least a portion of an immunoglobulin that is sufficient to confer specific antigen binding to the polypeptide. In certain embodiments, the antibody further comprises a label attached thereto and able to be detected (e.g., the label can be a radioisotope, fluorescent compound, enzyme or enzyme co-factor).

In certain embodiments, an antibody of the present disclosure is a monoclonal antibody, and in certain embodiments, the invention makes available methods for generating novel antibodies that specifically bind the biomarker or the exosome of the invention. For example, a method for generating a monoclonal antibody that specifically binds a biomarker or exosome, may comprise administering to a mouse an amount of an immunogenic composition comprising the biomarker or exosome, or fragment thereof, effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the biomarker or exosome. Once obtained, a hybridoma can be propagated in a cell culture, optionally in culture conditions where the hybridoma-derived cells produce the monoclonal antibody that binds specifically to the biomarker or exosome. The monoclonal antibody may be purified from the cell culture.

The term “specifically reactive with” as used in reference to an antibody is intended to mean, as is generally understood in the art, that the antibody is sufficiently selective between the antigen of interest (e.g., a biomarker or exosome) and other antigens that are not of interest. In certain methods employing the antibody, such as therapeutic applications, a higher degree of specificity in binding may be desirable. Monoclonal antibodies generally have a greater tendency (as compared to polyclonal antibodies) to discriminate effectively between the desired antigens and cross-reacting polypeptides. One characteristic that influences the specificity of an antibody: antigen interaction is the affinity of the antibody for the antigen. Although the desired specificity may be reached with a range of different affinities, generally preferred antibodies will have an affinity (a dissociation constant) of about 10-6, 10-7, 10-8, 10-9 or less.

Antibodies can be generated to bind specifically to an epitope of an exosome or a biomarker of the present disclosure, including, for example, neuron-derived exosomes, astrocyte-derived exosomes, oligodendrocyte-derived exosomes, and microglia-derived exosomes, or neuron-specific proteins selected from the group consisting of synaptosome associated protein 25 (SNAP25), neurogranin (NRGN), tau, phosphorylated tar, and synaptophysin, astrocyte-specific proteins selected from the group consisting of glial fibrillary acidic protein (GFAP) and excitatory amino acid transporter 1 (EAAT1), and oligodendrocyte-specific proteins selected from the group consisting of myelin basic protein (MBP) and oligodendrocyte myelin glycoprotein (OMG), a microglia-specific protein (CD11b), and chemokine (CX3CL1) or cytokine (IL1b, IL34, FasL, or IL12B). In another embodiment, the antibody generated is an anti-CD171 antibody, an anti-synaptosome associated protein 25 (SNAP25) antibody, an anti-neurogranin (NRGN) antibody, an anti-tau antibody, an anti-synaptophysin antibody, and anti-CD63 antibody, an anti-αβ-42 antibody, an anti-CD81 antibody, an anti-CTD antibody, an anti-GAPDH antibody, an anti-IL1b antibody, an anti-IL34 antibody, an anti-CX3CL1 antibody, an anti-glial fibrillary acidic protein (GFAP) antibody, an anti-excitatory amino acid transporter 1 (EAAT1) antibody, an anti-SNCA antibody, an anti-TH antibody, and anti-CD11b antibody, an anti-myelin basic protein (MBP) antibody, an anti-oligodendrocyte myelin glycoprotein (OMG) antibody, an anti-dopamine transporter (DAT) antibody, an anti-AchE antibody AchE, an anti-LAMP1 antibody LAMP1, an anti-REST antibody REST, an anti-SYT antibody SYT, an anti-SYP antibody, an anti-SYNPO antibody, an anti-PSD95 antibody, an anti-SV2A antibody, an anti-CCL2 antibody CCL2, an anti-IL34 antibody IL34, an anti-GYS antibody GYS, an anti-OR antibody OR, an anti-DR6 antibody DR6, an anti-HSP antibody HSP, an anti-IL12b antibody IL12b, an anti-Aβ antibody Aβ, or an anti-BACE antibody BACE.

In addition, the techniques used to screen antibodies in order to identify a desirable antibody may influence the properties of the antibody obtained. A variety of different techniques are available for testing interaction between antibodies and antigens to identify particularly desirable antibodies. Such techniques include ELISAs, surface plasmon resonance binding assays (e.g., the Biacore binding assay, Biacore AB, Uppsala, Sweden), sandwich assays (e.g., the paramagnetic bead system of IGEN International, Inc., Gaithersburg, Md.), western blots, immunoprecipitation assays, immunocytochemistry, and immunohistochemistry.

In some embodiments, the present disclosure relates to compositions used for identifying or treating asymptomatic brain trauma, including subconcussions. As detailed elsewhere herein, the present disclosure is based upon the findings that the levels of CD81, GAPDH, CTSD, NRGN, MBP, GFAP, Tau, phosphorylated Tau (e.g., T181), synaptophysin, CD63, αβ-42, SNCA, CX3CL1, IL1b, IL34, AchE, LAMP1, REST, SYT, TH, SYP, SYNPO, PSD95, SV2A, CCL2, IL34, GYS, OR, DR6, HSP, IL12b, Aβ, and/or BACE are implicated in the pathology of asymptomatic brain trauma.

In some embodiments, biomarkers inside vesicles are analyzed in addition to the surface biomarkers. In certain embodiments, the present disclosure relates to compositions for lysing vesicles (e.g., exosomes) in biological samples obtained from a subject. Lytic agents useful in the methods of the present invention include: RIPA buffer; Tris-HCl (pH 6.8); glycerol; SDS; 2-mercaptoethanol; Triton-X 100; M-PER Reagent; T-PER solution; and CHAPS. Lytic agents may be incubated with biological samples to disrupt the membrane of the vesicles of the present invention and release vesicle cargo (e.g., exosomal proteins) for subsequent analysis.

At the time of head trauma, the pliability of neuronal cell bodies enables them to withstand mild mechanical forces, whereas axons, shielded by the protective myelin sheath, exhibit greater rigidity, rendering them more vulnerable to compression, stretch, squeeze, crack, etc. (22-23) (FIG. 2B). Neurons detect such axonal problems through the disruption of neuronal signal transduction, then release 3 primary factors, such as brain-derived neurotrophic factor (BDNF), ciliary neurotrophic factor (CNTF), and neureglin-1 (NRG1). These factors migrate to damaged axonal lesions, and bind to respective receptors (TrkB for BDNF, CNTFR for CNTF, and ErbB for NRG1) expressed on the surface of ODC (FIG. 2B) (20-23). Upon binding, ODC become fully activated, initiating the repair processes of the damaged myelin sheath (FIG. 2B). This crosstalk between neurons and ODC via these neurotrophic factors can be considered as the first and the primary cellular response after axonal injuries.

When BDNF, CNTF, and NRG1 migrate to damaged axonal lesions, where ODE exist, these factors not only binds to respective receptors on the surface of ODC, but also bind to their receptors expressed on ODE or ODE surface non-specifically (FIG. 2B). Once bound to receptors on ODC, these ligands are internalized into the cytosol. However, this process does not occur in ODE as they lack the capability for internalization. Therefore, by monitoring the quantitative changes of BDNF, NRG1, and CNTF on the surface of ODE, it may be possible to assess post-event cellular cascades by a simple blood test. This approach offers a promising avenue for non-invasive evaluation of head trauma, irrespective of symptomatic manifestation.

The brain is a highly sophisticated network organ consisting of neurons, ODC, astrocytes, microglia, endothelial cells, and various intercellular parenchymal proteins. Neurotrophic factors and respective receptors described above are not specific to neurons and ODC but also expressed and secreted by other cells. Therefore, post-concussion biology is very complex, and numerous cascades are playing multiple roles simultaneously and sequentially. Since some are essential and others are byproducts, we need to identify and focus on the mainstream.

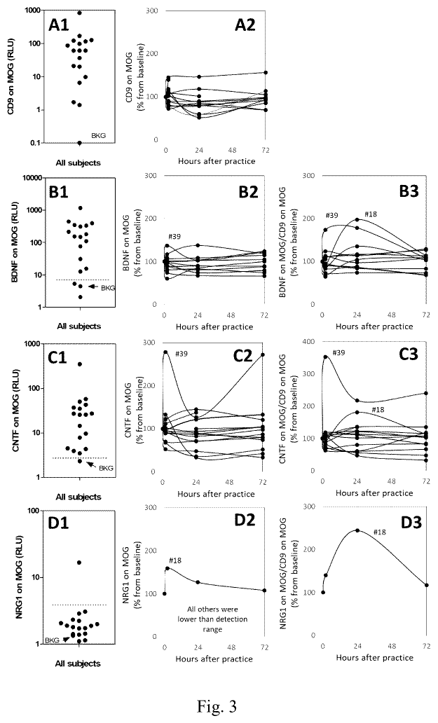

It is reasonable to speculate that axonal injury is the first and the primary event after mechanical head impact because of the rigidity of myelin sheath. While myelin is only produced by ODC in the brain, ODC must communicate with neurons to optimize and maintain the neuronal signal transduction. As shown in FIG. 2B, neuron-ODC crosstalk via neurotrophic factors is one of the most reasonable pathways. Since astrocytes and microglia also secrete all or some of these factors and express receptors (26-29), these cells also play important roles. For example, microglia are scavenger cells and clean unnecessary components from the lesion. Astrocytes are housekeepers to help these cellular functions. However, because non-ODC cells cannot synthesize myelin, they are supporting players in this situation. Thus, these cells are intentionally excluded from FIG. 2. If injury is very mild, neuron-ODC crosstalk alone may be sufficient for the cure. Case #18 may be the case. By switching anti-ODC to antibodies against neurons, astrocytes, microglia, and endothelial cells, and expanding probes beyond these 3 neurotrophic factors, many other reaction cascades can be assessed. This is practically feasible because the assay only requires a very small volume of plasma (5 μL in FIG. 3).

FIG. 3 shows preliminary data of soccer heading practice. Views A1 to D1: Plasma levels of CD9⁺MOG⁺ (A1), BDNF⁺MOG⁺ (B1), CNTF⁺MOG⁺ (C1), and NRG1⁺MOG⁺ (D1) as measured in an enzyme-linked immunosorbent assay (ELISA) with readings expressed as relative light units (RLU). Plasma samples were from 17 subjects before heading practice. Arrows: values of background (BKG) without plasma sample. Dotted lines: cutoff. Views A2 to D2: % from the baseline (before heading practice) of CD9⁺MOG⁺ (A2), BDNF⁺MOG⁺ (B2), CNTF⁺MOG⁺ (C2), and NRG1⁺MOG⁺ (D2), respectively. Views B3 to D3: B2-D2 data were normalized by the fluctuation of the amounts of ODE (A2).

BDNF, CNTF, NRG1, and various other biomarkers were extensively studied as blood markers of concussion and TBI. However, as described above, these factors are released from various cells to activate other cells and further released from these activated cells. Also, these factors are released into the bloodstream when the blood brain barrier (BBB) is disrupted. Thus, the increase of these blood biomarkers may continue for a while even after damages are fixed. In contrast, as shown in FIG. 3, case #18 showed transient increase at 24 hours, and return to the baseline levels in 72 hours. This may indicate that the ODC-based tests are directly corresponding to the repair of damaged axons. This further indicates that the tests may be applicable to the return-to-play decision. Because blood biomarkers are diluted in samples, sensitive assays are required, however, when ODC is isolated, ODC biomarkers can be detected by ordinary immunoassay.

As stated in the background section, the risk of symptom-free, asymptomatic or subconcussive brain injuries is well accepted in the public, and the demand of sensitive test is clearly growing. However, because we have no gold standard available for the assessment of such condition clinically, new tests cannot be validated easily. We can state that BDNF⁺MOG⁺, CNTF⁺MOG⁺, NRG1⁺MOG⁺signals are increasing after head impact and returning to the baseline levels for each athlete, but due to the absence of apparent symptoms and lack of clinical validation data, such information can be ignored. However, science moves one step at time, and the accumulation of new information opens the next chapter. Although conflict exists between public demand and difficulty of clinical validation, the concept (FIG. 2) and assay format (FIG. 3) provides insight into asymptomatic brain injury and a potential means to assess the outcome and impact of such injury.

### EXAMPLES

Contemplated and described embodiments will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications are intended to fall within the scope of the appended claims.

### Rationale

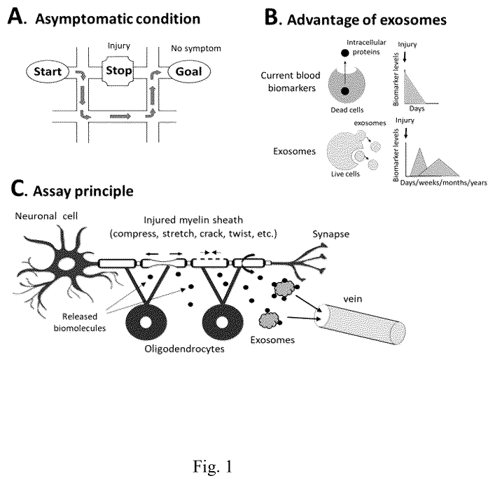

The pliability of neuronal cell bodies allows them to withstand mild mechanical forces, whereas axons-encased in a protective myelin sheath—are more rigid and therefore more vulnerable to damage from compression, stretching, squeezing, or cracking (FIG. 1C). As a result, axonal injuries are often considered the primary site of head trauma (30). Oligodendrocytes (ODCs) play a critical role in the repair process, as they are the only brain cells capable of producing myelin. During this process, various neurotrophic factors (NTFs) and inflammatory cytokines (ICs) are released at the injury site, promoting tissue recovery in mild cases or contributing to neuroinflammation in severe cases (FIG. 1C). Additionally, brain cells release exosomes or extracellular vesicles (EVs), which can migrate into the bloodstream (31, 15) (FIG. 1C). This presents a unique opportunity for non-invasive evaluation, similar to a liquid biopsy. As shown in FIG. 1B, exosome-based assays may be more sensitive than current blood tests because exosomes are actively released by live cells. In this study, we developed a technology to detect oligodendrocyte-derived exosomes (ODEs) in the blood. Our previous work demonstrated that exosomes can absorb various biomolecules on their surfaces (16). This suggests that by measuring locally released NTFs and ICs on the surface of ODEs in peripheral blood, we may be able to monitor the cascade of cellular and molecular events following axonal injury (FIG. 1B). While ICs are not tissue-specific, those found on ODEs can be directly associated with injured axons. For proof of concept, we selected three target biomarkers: ciliary neurotrophic factor (CNTF) as a recovery marker, interleukin 1B (IL1B) as a neuroinflammatory marker, and a-synuclein (SNCA) as a marker for Parkinson's disease (PD).

### Reagents

All reagents used in this study were consistent with those detailed in our previous publications (15,16), except for the following monoclonal antibodies: monoclonal antibody against human myelin oligodendrocyte glycoprotein (MOG) (clone JM23-06, Thermo Fisher Scientific, Waltham, MA), tropomyosin receptor kinase B (TRKB) (clone 72509, R&D Systems, Minneapolis, MN), CNTF (clone 09, MyBioSource, San Diego, CA), IL1B (clone H1b-98, BioLegend, San Diego, CA), and SNCA (clone 211, Thermo Fisher). All antibodies were commercially sourced and underwent appropriate validation through Western blot, immunohistochemistry, flow cytometry, and/or enzyme-linked immunosorbent assay (ELISA). Biotinylation of antibodies was performed using EZ-Link Sulfo-NHS-LC-Biotin (Thermo Fisher Scientific), followed by a spin column procedure to remove unbound biotin.

### Assay Protocol

The assay principle was a sandwich chemiluminescent ELISA with a combination of capture and detection antibodies. Protocol details were described in our previous report (15, 16). For this study, capture antibody was switched from anti-CD171 (neuron-specific marker) to anti-CD81 (exosome common marker), anti-MOG (ODC-specific), and appropriate control IgG (mouse or rabbit), with biotinylated probes against CD9 and CD81 (exosome markers), TRKB (ODC-specific marker), CNTF (brain recovery marker), IL1B (inflammatory cytokines), and SNCA (PD marker), respectively. These antibodies were carefully selected following extensive screening studies. Importantly, the captured exosomes remained intact, without lysis or permeabilization, to preserve their native structure and associated markers. After the probe reaction, each well was treated with streptavidin-horseradish peroxidase (Thermo Fisher Scientific), followed by a chemiluminescent substrate reaction using SuperSignal (Thermo Fisher Scientific). The relative light units (RLU) were measured using a luminometer (Active GLO, ANSH Labs, Webster, TX).

### Nanopartical Tracking Analysis (NTA)

To increase the assay volume, paramagnetic beads (PM-50, Spherotech, Lake Forest, IL) with surface characteristics identical to those of the ELISA wells were utilized. Anti-MOG was immobilized on the bead surface, and ODE capture was successfully validated. After capturing the ODEs, they were released from the beads by briefly exposing them to a pH 2.0 solution for less than 30 seconds. The supernatant was immediately transferred to a high-pH solution to neutralize the acidic environment. NTA of the released exosomes was performed by Particle Technology Labs (Downers Grove, IL).

### Example 1: Blood Test Method: Soccer Players

We have previously reported a unique blood test for the measurement of plasma levels of neuron-derived EV (NDE), by a sandwich immunoassay between antibodies against neurons (CD171 or L1 cell adhesion molecules (L1CAM)) and EV (CD9) (15). Through extensive optimization, we identified CD171⁺CD9⁺ double positive signals as indicative of NDE (15). Similarly, we have also developed another system against ODE (16-17). In these systems, the antibodies against ODC-specific proteins are used to capture ODE from plasma samples, then probed with antibodies against CD9 and other target molecules.

In this study, we applied as little as 5 μL of plasma to anti-myelin oligodendrocyte glycoprotein (MOG)-immobilized microplates to capture ODE, then probed with biotinylated antibodies against BDNF, CNTF, and NRG1, respectively, followed by the reaction with horseradish peroxidase-streptavidin conjugate, similar to our previous studies (15-17). Double positive signals of both MOG and 3 neurotrophic factors were determined by chemiluminescent reaction and relative light units (RLU) were measured by a luminometer.

Plasma samples were provided by our collaborator, Dr. Keisuke Kawata (Indiana University) and the aliquots were used in his (24) and our previous study (15). These samples were collected before, 2, 24, and 72 hours after intensive soccer heading practice by the protocol previously described (25) and subconcussive conditions were assessed by near-point of convergence (NPC), which measures the closest point of focus before diplopia occurs (24). Due to the nature of feasibility study, leftover samples of anonymous 17 athletes were used. Although subconcussive conditions were induced and assessed by NPC, all participants did not show any neurological problems. Thus, this is an ideal human study model for the biomarker discovery of acute phase of asymptomatic/subconcussive head impact. These samples were used to measure plasma levels of NDE in our previous publication, however, NDE levels did not show any meaningful changes (15).

As shown in FIG. 3B1, plasma levels of BDNF⁺MOG⁺double positive signals were also widely spread and 2 athletes were under detection limit. However, these values were quite stable in each individual (FIG. 3B2). After normalization by dividing each values with the value of ODE itself, #18 and #39 showed clear increase of BDNF⁺MOG⁺ 2-24 hours after head impact, and return to the baseline levels in 72 hours (FIG. 3B3). As shown in FIG. 3C1, plasma levels of CNTF⁺MOG⁺double positive signals were also widely spread and all athletes were higher than detection limit. Similar to the results of BDNF⁺MOG⁺, #18 showed a transient increase of CNTF⁺MOG⁺ at 24 hours, and returned to the baseline in 72 hours (FIG. 3C3), while the values of #39 were biphasic (FIG. 3C2) or sustained high for 72 hours (FIG. 3C3). This may indicate that the initial CNTF activates nearby cells and more CNTF are released from these cells to induce secondary reactions. This is reasonable because CNTR receptors are present in not only ODC, but also other glia cells (26-27). Anti-NRG1 used in this study was not sensitive and only 1 subject (#18) showed reasonable data (FIG. 3D1). As shown in FIG. 3D2 and FIG. 3D3, this subject showed similar pattern to those of BDNF and CNTF.

Since this was an innocent soccer heading practice project, we did not anticipate seeing any increases. While the number of samples was very small, we found athletes with stable (all except #18 and #39), transient (#18) and sustained increases (#39). Although the exactly the same head impact is applied, each person's brain susceptibility is not always identical. Thus, the variation among 17 athletes cannot be ignored. The assay sensitivity can be improved substantially by finding more sensitive and specific antibodies and applying much larger volume of plasma samples. For such sustained cases, we may be able to analyze biomarkers responsible for various secondary reaction pathways in future.

### Example 2: Blood Test Method: Additional Athletic Groups

Plasma samples—Control Group. Healthy adult ethylenediaminetetraacetic acid (EDTA)-plasma samples were purchased from 3 different commercial sources (Innovative Research, Novi, MI, BioIVT, Westbury, NY, and Equitech Enterprise, Kerrville, TX).

Professional boxers and mixed martial arts (MMA) fighters. Following approval by the California State Athletic Commission (CSAC) and Institutional Review Board (IRB) (Pearl IRB, Indianapolis, IN), professional boxers and MMA fighters (14 participants with 4 repeated measures, total 18 samples, all male; Table I) were recruited for this study. Recruitment occurred during events organized by SOCA Fights (Fountain Valley, CA). The CSAC, which oversees professional and amateur boxing, kickboxing, and MMA across California, serves as a model for the safety and fairness of regulated sporting events. Baseline blood samples were collected during weigh-ins one day before the bout. Subsequent samples were taken after the bout, the next day, and at one, two, and five weeks post-bout. Capillary blood samples were collected using the Tasso Kit (Seattle, WA), with plasma isolated on the same day as collection. Aliquots were stored in a −80° C. freezer. Concussion symptoms were documented via questionnaire. Fights were closely reviewed through pay-per-view broadcasts on TrillerTV (New York, NY).

TABLE 1

| | | | | | | Bout information | | | | | | | | |
|---|---|---|---|---|---|---|---|---|---|---|---|---|---|---|
| | | | | Record | # of | Boxing/ | | | | Symptoms** | | | | CNTF on |
| # | Age | Ethnicity | Repeat | (W-L-D)* | KO | MMA | Class | Results | Lost by KO | D1 | W1 | W2 | W5 | MOG |
| #1 | 18 | Latino | | 1-0-0 | 0 | Boxing | Welterweight | Won by Decision | | (−) | (−) | (−) | (−) | |
| #2 | 20 | Latino | | 3-0-0 | 0 | Boxing | Super Lightweight | Won by Decision | | (−) | (−) | (−) | (−) | |
| | | | 2nd | 4-0-0 | 0 | Boxing | Super Lightweight | Won by Decision | | (−) | (−) | (−) | (−) | |
| #3 | 24 | Black | | Pro debut | 0 | Boxing | Lightweight | Won by KO | | (−) | (−) | (−) | (−) | |
| | | | 2nd | 1-0-1 | 0 | Boxing | Lightweight | Won by Decision | | (−) | (−) | (−) | (−) | |
| #4 | 25 | Latino | | 5-0-1 | 0 | Boxing | Light Heavyweight | Won by KO | | (−) | (−) | (−) | (−) | |
| | | | 2nd | 6-0-1 | 0 | Boxing | Light Heavyweight | Won by Decision | | (−) | (−) | (−) | (−) | |
| | | | 3rd | 7-0-1 | 0 | Boxing | Super Middleweight | Won by Decision | | (−) | (−) | (−) | (−) | |
| #5 | 25 | Latino | | [symbol] 2-1-0 | 0 | Boxing | Super Welterweight | Won by Decision | | (−) | (−) | (−) | (−) | |
| #6 | 25 | Black | | Pro debut | 0 | Boxing | Welterweight | Lost bt decision | | (−) | (−) | (−) | (−) | |
| #7 | 28 | Latino | | 3-0-0 | 0 | Boxing | Lightweight | Won by KO | | (−) | (−) | (−) | (−) | |
| #8 | 28 | Latino | | Pro debut | 0 | Boxing | Super Middleweight | Lost bt decision | | (−) | (−) | (−) | (−) | |
| #9 | 31 | Black | | 0-0-1 | 0 | Boxing | Flyweight | Łosi by KO | Head punch | (−) | (−) | (−) | (−) | |
| #10 | 32 | Latino | | [symbol] 2-6-1 | 1 | MMA | Bantamweight | Won by KO | | (−) | (−) | (−) | (−) | |
| #11 | 32 | Caucasian | | [symbol] 5-5-0 | 1 | MMA | Bantamweight | Lost by KO | Choke | (−) | (−) | (−) | (−) | |
| #12 | 35 | Black | | 0-1-0 | 1 | Boxing | Lightweight | Lost by KO | Head punch | (+) | (−) | (−) | (−) | Elevated |
| #13 | 36 | Black | | [symbol] 7-1-1 | 0 | MAMA | Lightweight | Lost by Ko | Choke | (−) | (−) | (−) | (−) | |
| #14 | 43 | Caucasian | | 9-0-0 | 0 | Boxing | Heavyweiht | Won by KO | | (+) | (+) | (−) | (−) | |
| | | | 2nd | 10-0-0 | 0 | Boxing | Heavyweiht | Lost by Ko | Head punch | (+) | (+) | (−) | (−) | Elevated |

*W-L-D: Won-Lost-Draw
**Concussion symptoms [D1: next day, W1-5: 1-5 weeks after bout]

High school athletes. Frozen archived plasma samples from 137 American football players (all male) at six different high schools were analyzed in this study. Blood samples were collected monthly from pre-season (July) to post-season (November), yielding a total of 919 samples. Frozen archived plasma from 68 cross-country athletes (all male) served as controls given less prevalence of head injuries in this population, with blood collection timed similarly to that of the football players. All samples were approved for use by the Indiana University IRB (Bloomington, IN) and had been analyzed in a prior study (15).

College rugby players. Frozen archived plasma samples from 14 college rugby players (all male) were included in this study. Plasma samples (n=138) were collected 3-4 times per year, regardless of concussion history. Ethical approval was granted by the Ethics Committee of Keio University (Kanagawa, Japan).

Validation of antibody capture. MOG, a transmembrane protein primarily expressed on ODCs, has an extracellular domain recognized by the antibody used in this study. The first experiment compared anti-MOG (rabbit IgG) and control rabbit IgG immobilized on ELISA wells using seven different control plasma samples and phosphate-buffered saline (PBS) as a negative control.

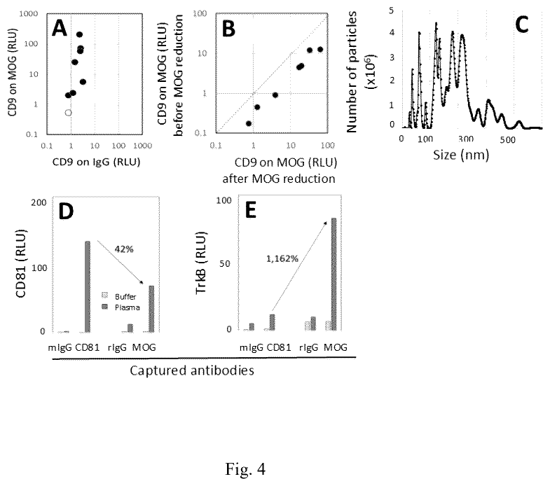

FIG. 4 shows validation of capture antibody. A. MOG-specificity. Seven different control plasma samples (•) and phosphate-buffered saline (PBS) (∘) were applied to ELISA wells coated with anti-MOG (Y-axis) and control rabbit IgG (X-axis). After washing away unbound materials, the wells were exposed to biotinylated anti-CD9 probes to quantify the captured CD9⁺ exosomes. The assay details are described in the Methods section. B. MOG reduction. Seven control plasma samples and a PBS sample were applied to the first anti-MOG-coated ELISA wells to capture MOG⁺ exosomes. The supernatants were then transferred to a second anti-MOG-coated well to capture any remaining MOG⁺ exosomes. Both wells were probed with anti-CD9 to quantify the amounts of captured CD9⁺ exosomes. To normalize the data, plasma values were subtracted by PBS values. The Y-axis represents the results from the first wells (pre-reduction), and the X-axis represents the results from the second wells (post-reduction). C. Nanoparticle tracking analysis (NTA). Captured exosomes on anti-MOG-immobilized paramagnetic beads were eluted and analyzed using NTA to confirm the presence and size distribution of exosomes. Assay details are described in the Methods section. The X-axis represents the particle size, and the Y-axis represents the number of particles detected at each size. D. Enrichment of ODEs. Control plasma and buffer (PBS) were applied to mouse IgG (mIgG) and anti-CD81 (exosome marker, mouse IgG) immobilized ELISA wells for the capture of whole exosomes, as well as rabbit IgG (rIgG) and anti-MOG (rabbit IgG) immobilized ELISA wells for the capture of ODEs. The first set of wells was probed with anti-CD81 to quantify the captured exosomes (D), and the second set was probed with anti-TRKB to quantify ODEs (E). The amounts of CD81⁺ whole exosomes captured in anti-MOG wells were 42% of those captured in anti-CD81 wells (D), whereas the amounts of TRKB⁺ ODE in anti-MOG wells were 1,162% of those captured in anti-CD81 wells (E).

As shown in FIG. 4A, the CD9 probe (an exosome marker) produced substantially higher signals in the anti-MOG wells ([CD9 on MOG], Y-axis) compared to control IgG wells ([CD9 on MOG], X-axis). Minimal CD9 signal was observed in the PBS control wells (FIG. 4A ∘). Similar to our prior work on neuron-derived exosomes, [CD9 on MOG] values (Y-axis, log scale) exhibited a wide individual-to-individual variability (FIG. 4A, right panel).

The second experiment aimed to deplete MOG⁺ exosomes from plasma. Seven control plasma samples and a PBS sample were applied to anti-MOG wells for exosome capture. The supernatants were then transferred to a second set of anti-MOG wells to capture any remaining MOG⁺ exosomes. Both sets of wells were probed with anti-CD9 to quantify the amounts of captured CD9⁺ exosomes. Data were normalized by subtracting PBS values (n=7; FIG. 4B). On the Y-axis are CD9⁺ exosome levels before depletion (1st wells), and on the X-axis are levels after depletion (2nd wells). As shown in FIG. 4B, CD9⁺ exosome quantities were reduced in the second wells (below the 45° line), confirming the effectiveness of anti-MOG in capturing MOG⁺ exosomes.

The third experiment focused on measuring exosome counts and size distribution. Due to the small surface area of ELISA wells, paramagnetic beads with identical surface properties were used to increase exosome yield (see Methods for details). NTA confirmed that anti-MOG captured exosomes with sizes ranging from 100-300 nm (FIG. 4C).

The fourth experiment assessed the enrichment of ODEs in anti-MOG wells. First, we confirmed that anti-MOG captured exosomes were reacted with TRKB, one of ODC-specific marker. Then, control plasma and buffer (PBS) were applied to mouse IgG (mIgG) and anti-CD81 (exosome marker, mouse IgG) immobilized ELISA wells for the capture of whole exosomes, as well as rabbit IgG (rIgG) and anti-MOG (rabbit IgG) immobilized ELISA wells for the capture of ODEs. Then the 1st set was probed with anti-CD81 to quantify the captured exosomes (FIG. 4D) and the 2nd set was probed with anti-TRKB to quantify ODEs (FIG. 4E). The anti-MOG wells captured 42% of the CD81⁺ whole exosomes compared to anti-CD81 wells (FIG. 4D). However, the amount of TRKB⁺ ODEs in anti-MOG wells was 1,162% of the levels captured in anti-CD81 wells (FIG. 4E), indicating that anti-MOG enriched ODEs by more than 20-fold compared to whole exosome capture.

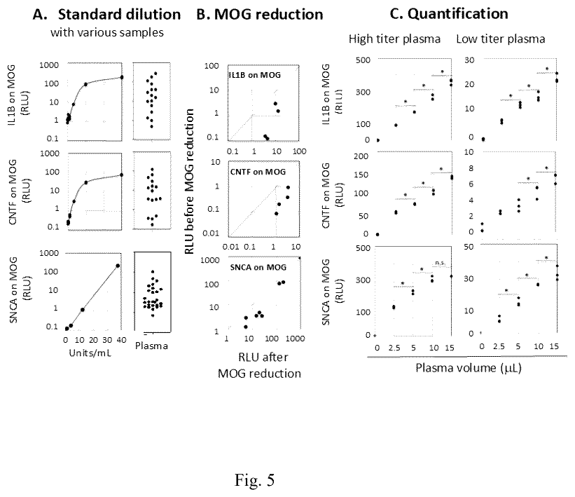

Validation of probes. A. Standard plasma dilution and the results of various control plasma samples. Our standard plasma was assigned to 100 units/mL, and dilution study was conducted for IL1B (top), CNTF (middle), and SNCA (bottom), respectively. All showed linear increase with (IL1B and CNTF) or without saturation (SNCA). Similar to [CD9 on MOG] (FIG. 4A right), each value was widely different among subjects (FIG. 5A right). B. MOG reduction. The quantity of MOG⁺ ODEs was reduced as shown as FIG. 4B. The quantity of [IL1B on MOG] (top), [CNTF on MOG] (middle), and [SNCA on MOG] (bottom) all reduced after MOG depletion, indicating that measured IL1B, CNTF, and SNCA were MOG dependent. C. Quantification. As shown in FIG. 5A (right panel), the levels of IL1B, CNTF, and SNCA exhibited substantial inter-individual variability among subjects. This variability indicates that absolute biomarker values are not suitable for cross-sectional studies aimed at diagnosing concussive or subconcussive conditions.

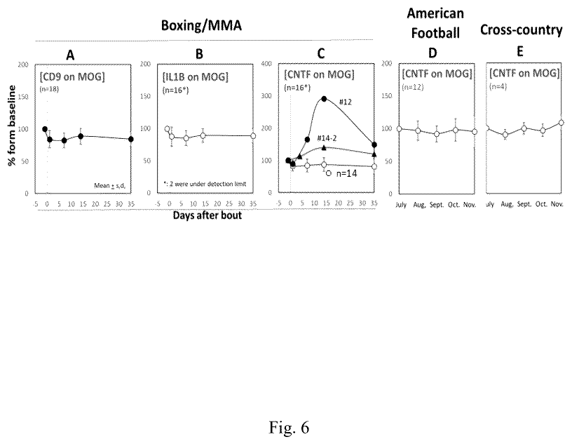

However, the values were highly consistent within each individual over time (FIGS. 6-9), suggesting that subject-specific changes are more reliable for analysis. In FIG. 5C, plasma samples with high (FIG. 5C, left) and low (FIG. 5C, right) titers were applied to anti-MOG wells in volumes of 0, 2.5, 5, 10, and 15 μL, in triplicate, and probed with antibodies targeting IL1B (top), CNTF (middle), and SNCA (bottom), respectively. The results demonstrate minimal variation among triplicates, and even small changes in plasma volume (50-100%) resulted in measurable differences. Given this sensitivity, we established a threshold for significant biomarker changes. For subsequent athlete studies, we focused on individuals who exhibited greater than 100% change from their baseline levels (i.e., >200% of baseline values). To save the precious clinical samples, plasma samples were used in singlicate.

Results of brain recovery marker, CNTF—Boxing and MMA. These two sports provide an ideal human research model for assessing concussion and subconcussive brain injuries, because blood samples can be collected before the bout, and head impacts can be monitored in real time via broadcasted video. In the SOCA events, all participants were professional fighters, with half being local and the other half visitors. For the subsequent 5-week follow-up, we focused on recruiting only local fighters. Blood draws were performed at regular intervals, accompanied by concussion symptom assessments via questionnaires. The incidence of knockout (KO) in these combat sports is notably high. Out of 18 fighters, five were lost by KO—two by rear-naked choke in MMA and three by head punches in boxing (Table I).

FIG. 6 shows results of [CNTF on MOG]. A-C: Boxers and MMA fighters (n=18 in A, n=16 in B-C (2 were under detection limits). D: American football players (n=12). E: Cross-country athletes (n=4). A: [CD9 on MOG]. B: [IL1B on MOG]. C-E: [CNTF on MOG]. Symbols (∘) and bars were mean±standard deviation of whole subject (A, B, D, E) or stable subjects only (n=14, C). 2 individual data were shown in C•. X axis in A-C: before and after the bout. D-E: Blood collection once a month from July to November. Y axis: % from baseline (1^{st} blood collection in each subject).

Surprisingly, only three of these fighters showed concussion symptoms (Table I). As shown in FIGS. 6A-6B, ODE levels ([CD9 on MOG]) and [IL1B on MOG] did not show substantial changes post-bout. Similarly, in FIG. 6C, the majority of fighters exhibited no significant increase in [CNTF on MOG]. However, two fighters who lost by KO from clean head punches (Table I) showed an increase in [CNTF on MOG] around 1-2 weeks after the bout, with levels returning to baseline by 5 weeks. According to the concussion questionnaire, these two fighters experienced concussion symptoms (headache, dizziness, and sleep problems) post-bout, which completely resolved within 2 weeks, before the peak [CNTF on MOG] levels. We also analyzed plasma samples from high school American football players (n=12) and cross-country athletes (n=4). [CNTF on MOG] levels were stable from July to November in these cohorts. In summary, those boxers who were cleanly knocked out exhibited a peak in CNTF on MOG levels, indicative of hidden brain damage and recovery, at a time when their signs and self-reported symptoms had dissipated. Although the number of positive fighters was small, these data provide encouraging insights to advance our research further with larger sample sizes.

Results of neuroinflammation marker, IL1B—American football. Repetitive mild head impacts are considered a potential risk factor for long-term neurological consequences. However, due to the lack of overt symptoms and the absence of appropriate objective assessment tools, these conditions are often overlooked. Blood samples collected from American football players provide an ideal model for studying the effects of repetitive head impacts.

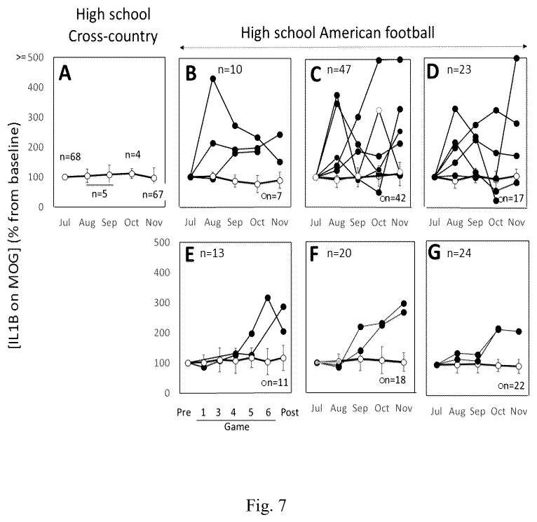

FIG. 7 shows results of [IL1B on MOG]—High school cross-country and American football. A: High school cross-country athletes. B-G: American football players from 6 different high schools (n=10, 47, 23, 13, 20, and 24 in each school, total 137 subjects, 919 plasma samples). X axis: Blood collection once a month from July to November (A-G except E), and before each game and pre-July) and post-season (November) in E. Y axis: [IL1B on MOG], % from baseline (1st blood collection in each subject). Symbols (∘) and bars are mean±standard deviation of whole subject (A) or stable subjects only (B-E). Subjects with >200% are shown individually in B-E•.

As shown in FIG. 7A, [IL1B on MOG] levels were very stable from July to November in cross-country athletes, serving as a model for non-contact control sports from the same high schools as the American football players. Due to variations in protocols between each school, we present the data from six different high schools individually (FIG. 7B-7G) In each school, the majority of athletes (American football) showed no substantial increase in [IL1B on MOG] (mean (∘), standard deviation (bar), and the number (n) of stable athletes in each panel). However, 19 athletes showed an increase greater than 200% (i.e., a twofold increase from preseason values) during the sports season, from August to November (FIG. 7B-7G •). Interestingly, some athletes exhibited an immediate rise in August, which gradually returned to baseline levels, while others showed a gradual increase toward the end of the season. Additionally, school-to-school variation was observed. Schools B and D showed early increases in [IL1B on MOG] in August, possibly indicating more intense practices from the start of the season. In contrast, Schools E-G exhibited a gradual increase in [IL1B on MOG] levels toward the end of the season, suggesting that the intensity of practices increased over time. School C appeared to have the most intense practices. It is important to note that the increase in [IL1B on MOG] was not permanent, and levels returned to baseline over time. Most importantly, none of the athletes showed concussion symptoms, indicating that [IL1B on MOG] may serve as a sensitive biomarker for detecting subclinical TBI resulting from innocuous or milder subconcussions.

### Results of Neuroinflammation Marker, IL1B—Rugby.

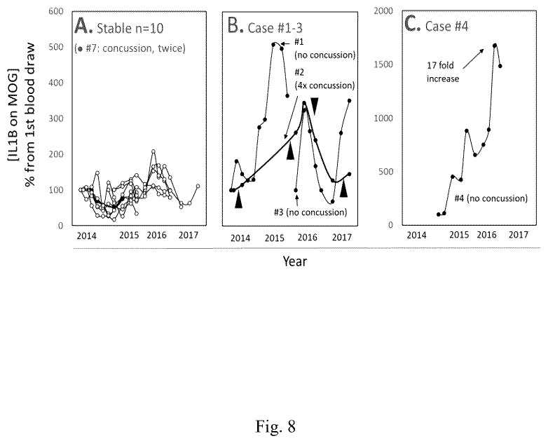

FIG. 8 shows results of [IL1B on MOG]—College rugby. A: Rugby players who did not show >200% (n=10). Since start date is not uniform, each subject data is shown. Nine were no concussion and 1 showed concussion twice (solid line). B. Case #1-3. Case #1 and 3 showed no concussion, and #2 showed concussion 4 times as indicated by solid triangles. C. Case #4 with different y-axis scale. Y axis: [IL1B on MOG], % from baseline (1st blood collection in each subject).

As shown in FIG. 8A, the majority (n=10) of rugby players in our sample (n=14) exhibited stable [IL1B on MOG] levels over the 4-year period from 2014 to 2017. However, three players (#1-3) demonstrated a greater than twofold increase during this time (FIG. 8B). Player #1 showed stable levels in 2014, but experienced a gradual increase, reaching a fivefold rise during the 2015 season. Player #2, who had suffered four concussions (A) during this period, was stable in 2014, but showed an increase in [IL1B on MOG] in 2016, returning to baseline in 2017. Player #3 showed an increase in [IL1B on MOG] in 2016, which returned to baseline by the end of the year, followed by a similar increase in the 2017 season. The most striking case was Player #4 (FIG. 8C), where [IL1B on MOG] levels gradually increased from 2015 to 2016, reaching more than a 17-fold increase by the end of his college career. Interestingly, players #1, #3, and #4 did not experience symptomatic concussions during the study period.

Results of SNCA-long-term PD risk. Repetitive head impacts are recognized as a potential risk factor for developing PD later in life (32), with notable examples including boxing legend Muhammad Ali and retired NFL quarterback Brett Favre. A key pathological feature of PD is the accumulation of SNCA in the brain (33). Intracellular SNCA aggregation forms Lewy bodies, a hallmark of PD, which contribute to neuron-specific toxicity (34). Extracellular SNCA accumulation, however, may promote disease propagation by spreading and seeding aggregation in otherwise healthy neurons (35). This extracellular SNCA, released around axonal injury sites, may bind to the surface of ODEs. To investigate this hypothesis, we measured [SNCA on MOG] in various athlete samples.

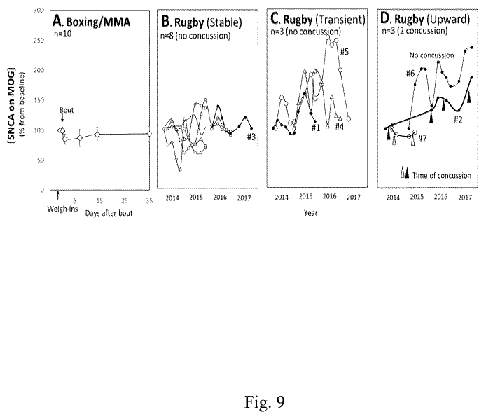

FIG. 9 shows results of [SNCA on MOG]. A: Boxers and MMA fighters. Symbols (∘) and bars are mean±standard deviation of all subjects (n=10). B. Rugby players who showed stable [SNCA on MOG] values (n=8). Subject #3 (•), who showed an increase in [IL1B on MOG] in FIG. 8, was included in this group. C. Rugby players who showed a transient >150% increase in [SNCA on MOG]. Subject #1 and #4 showed an increase in [IL1B on MOG] (as shown in FIG. 8), while subject #5 did not. None of the players showed concussion symptoms. D. Rugby players who showed a continuous upward trend of [SNCA on MOG] (subject #2 and #6) and no increase despite two concussion episodes (subject #7). Subject #2 experienced 4 concussions and showed an increase in [IL1B on MOG] (FIG. 8). Subject #6 had no concussions and showed no increase in [IL1B on MOG]. The time of concussion is shown in open and solid triangles.

As shown in FIG. 9A, no increase in [SNCA on MOG] was observed following boxing or MMA events. FIG. 9B shows that the majority (n=8/14) of rugby players who did not report concussion exhibited stable [SNCA on MOG] levels. Notably, Player #3, who showed an increase in [IL1B on MOG] (FIG. 8B), did not exhibit any increase in [SNCA on MOG]. In FIG. 9C, three players (#1, 4, and 5) showed a transient increase in [SNCA on MOG], but these values returned to baseline by the end of the study period. Player #1 and Player #4 also showed an increase in [IL1B on MOG] (FIGS. 8B and 8C). Player #5 demonstrated an increase in [SNCA on MOG], but did not show an increase in [IL1B on MOG] (FIG. 8A). In FIG. 9D, two players (Player #2 and #6) showed a persistent increase in [SNCA on MOG], which continued throughout the research period. Interestingly, Player #6, despite showing a persistent [SNCA on MOG] increase, did not report a concussion and did not show any increase in [IL1B on MOG]. Player #7, who reported two concussions (Δ), showed no increase in either [IL1B on MOG] (FIG. 8A) or [SNCA on MOG] (FIG. 9D). In summary, 5 of our 14 rugby players showed elevations in the [SCNA on MOG] over a period of 4 years. These findings highlight that [IL1B on MOG] and [SNCA on MOG] are independent parameters, and their changes do not necessarily correlate with each other or with reported concussion events.

As discussed throughout this disclosure, subconcussion remains an elusive concept in medical terminology, lacking clear and practical diagnostic criteria. While several protocols exist for assessing concussion and its recovery—such as the Post-Concussion and Cognitive Testing (ImPACT) (36), Acute Concussion Evaluation (ACE) (37), Sports Concussion Assessment Tool (SCAT) (38), Balance Error Scoring System (BESS) (39), the Graduated Return To Play (GRTP) protocol (40), and Team Physician Consensus Statement of Return to Sport/Return to Play (41)—the results are often subjective. Athlete's symptom manifestation play a significant role in the assessment, making the process less objective. Notably, there are currently no biological or molecular tests to guide decisions regarding the return-to-play for athletes, combat sports fighters, or military personnel. In the absence of such tools, physical and cognitive assessments are conducted. However, these tests are often limited in scope, as they primarily focus on specific neuronal areas, potentially overlooking injuries to other regions of the brain. In contrast, our blood-based test offers a more comprehensive approach, applicable to any damaged axonal lesions, because MOG is common to all axons. While the mechanical strength of head impacts can be measured using motion sensors, these impacts do not necessarily induce identical brain responses, and the variation in individual responses is considerable. This study represents vital pioneering work to tackle this complex issue, and although many questions remain unanswered, the three blood biomarkers described in this report (measuring [CD9 on MOG], [IL1B on MOG], and [SNCA on MOG]) were well-characterized (FIGS. 4-5) and successfully applied in various sporting events (FIGS. 6-9). The assay platform used is a standard ELISA, requiring no specialized devices or instruments, and only a small volume of venous or capillary blood is needed. Furthermore, as demonstrated in FIGS. 7-9, even frozen archived plasma samples can be utilized for the test. Thus, the approach outlined in this report may serve as a critical step forward in monitoring athletes' brain health in the future.

When the myelin sheath is weakly damaged, neuronal cells detect the impairment in neurotransmission and release NTFs such as nerve growth factor (NGF) and brain-derived neurotrophic factor (BDNF). These factors bind to their respective receptors—tropomyosin receptor kinase A (TRKA) and TRKB—which are expressed on the surface of ODCs (42). This initial cross-talk between neuronal cells and ODCs is one of the first steps in the repair process. Activated ODCs release BDNF, which in turn activates surrounding neuronal cells to extend axons and establish new neural networks. Astrocytes also sense the damage and respond by secreting BDNF, CNTF, and glial cell-derived neurotrophic factor (GDNF), along with various pro- and anti-inflammatory cytokines. These factors activate microglia, which either support the repair process or contribute to neuroinflammation, depending on the context. When axons are disrupted, the distal portion undergoes Wallerian degeneration (43). Microglia play a critical role in cleaning up the resulting debris by releasing various NTFs and ICs. While these processes are conceptual, they may vary between individuals. This study provides new insights into the complex post-injury repair processes and could lead to a better understanding of how these mechanisms unfold in athletes following head impacts.

Data presented herein showed that test values were reversible, often returning to baseline levels. This suggests that temporarily halting activity until these levels normalize could be a prudent approach to prevent further complications. However, such decisions should be made collaboratively by athletes, coaches, team doctors, and families. Moreover, these findings could contribute to the improvement of safety protocols and promote the development of sports equipment, medications, and other interventions designed to reduce the risks associated with head impacts and accelerate recovery processes.

### Abbreviations

CDC: Centers for Disease Control and Prevention, TBI: traumatic brain injuries, CT: Computed Tomography, MRI: Magnetic Resonance Imaging, UCH-L1: ubiquitin C-terminal hydrolase L1, GFAP: glial fibrillary acidic protein, CTE: chronic traumatic encephalopathy, PD: Parkinson's disease, ODC: Oligodendrocytes, NTFs: neurotrophic factors, ICs: inflammatory cytokines, EVs: extracellular vesicles, ODEs: ODC-derived EVs, CNTF: ciliary neurotrophic factor, IL1B: interleukin 1B, SNCA: a-synuclein, MOG: myelin oligodendrocyte glycoprotein, TRKB: tropomyosin receptor kinase B, ELISA: enzyme-linked immunosorbent assay, RLU: relative light units, NTA: nanoparticle tracking analysis, EDTA: ethylenediaminetetraacetic acid, MMA: mixed martial arts, CSAC: California State Athletic Commission, IRB: Institutional Review Board, PBS: plasma and buffer, mIgG: mouse IgG, rIgG: rabbit IgG, KO: knockout, NFL: National Football League, ImPACT: Immediate Post-Concussion and Cognitive Testing, ACE: Acute Concussion Evaluation, SCAT: Sports Concussion Assessment Tool, BESS: Balance Error Scoring System, GRTP: Graduated Return To Play protocol, NGF: nerve growth factor, BDNF: brain-derived neurotrophic factor, TRKA: tropomyosin receptor kinase A, GDNF: glial cell derived neurotrophic factor, AD: Alzheimer's disease, MRS: magnetic resonance spectroscopy, PET: positron emission tomography, WHO: World Health Organization, PCS: post-concussion syndrome; PTSD: post-traumatic stress disorder; SAC: Standardized Assessment of Concussion, BDNF: brain-derived neurotrophic factor; NRG1: neureglin-1; NDE: neuron-derived EV (NDE), L1CAM: L1 cell adhesion molecules; RLU: relative light units; BBB: blood brain barrier.

Thus, specific embodiments, methods of blood biomarkers of oligodendrocyte-derived exosomes and their use in identifying asymptomatic brain injury, including subconcussive brain injury have been disclosed. It should be apparent, however, to those skilled in the art that many more modifications besides those already described are possible without departing from the inventive concepts herein. The inventive subject matter, therefore, is not to be restricted except in the spirit of the disclosure herein. Moreover, in interpreting the specification and claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

### REFERENCES

1. WHO. https://www.who.int/publications/i/item/rehabilitation-for-persons-with-traumatic-brain-injury (accessed Mar. 18, 2024).
2. C. Cancelliere, L. Verville, J. L. Stubbs, H. Yu, C. A. Hincapié, J. D. Cassidy, J. J. Wong, H. M. Shearer, G. Connell, D. Southerst, S. Howitt, B. Guist, N. D. Silverberg, Post-Concussion Symptoms and Disability in Adults With Mild Traumatic Brain Injury: A Systematic Review and Meta-Analysis. J. Neurotrauma. 40 (2023) 1045-1059.
3. L. Papa, M. R. Zonfrillo, R. D. Welch, L. M. Lewis, C. F. Braga, C. N. Tan C N, N. J. Ameli, M. A. Lopez, C. A. Haeussler, D. Mendez Giordano, P. A. Giordano, J. Ramirez, M. K. Mittal, Evaluating glial and neuronal blood biomarkers GFAP and UCH-L1 as gradients of brain injury in concussive, subconcussive and non-concussive trauma: a prospective cohort study. BMJ Paediatr. Open. 3 (2019) e000473.
4. M. McCrea, J. P. Kelly, J. Kluge, B. Ackley, C. Randolph, Standardized assessment of concussion in football players. Neurology. 48 (1997) 586-8.
5. C. Bailey, J. Meyer, D. Soden, C. Tangen, R. Deane, S. Briskin, J. Gunstad, P. Fastenau, P. Smith, M. Solomon, A. W. Kelly. SCAT5 Sex Differences: Normative Data, Clinical Thresholds, and Relevance for Identification of Concussion. Arch. Clin. Neuropsychol. 37 (2022) 1536-1544.
6. K. G. Quigley, M. R. Taylor, D. Hopfe, P. Pavilionis, N. G. Murray, Minimal detectable change for the ImPACT test administered remotely. J. Athl. Train. 58 (2023) 981-986.
7. B. Omalu, Chronic traumatic encephalopathy. Prog. Neurol. Surg. 28 (2014) 38-49.
8. J. E. Bailes, A. L. Petraglia, B. I. Omalu, E. Nauman, T. Talavage, Role of subconcussion in repetitive mild traumatic brain injury. J. Neurosurg. 119 (2013) 1235-45.
9. J. E. Resch, A. Rach, S. Walton, D. K. Broshek, Sport Concussion and the Female Athlete. Clin. Sports Med. 36 (2017) 717-739.
10. S. Rowson, M. W. Goforth, D. Dietter, P. G. Brolinson, S. M. Duma, Correlating cumulative sub-concussive head impacts in football with player performance-biomed 2009. Biomed. Sci. Instrum. 45 (2009) 113-8.
11. Cdc.gov/HEADSUP
12. Nowinski C J, Rhim H C, Mckee A C, et al. ‘Subconcussive’ is a dangerous misnomer: hits of greater magnitude than concussive impacts may not cause symptoms. Br J Sports Med. 2024; 58 (14): 754-756.
13. Bazarian J J, Biberthaler P, Welch R D, et al. Serum GFAP and UCH-L1 for prediction of absence of intracranial injuries on head CT (ALERT-TBI): a multicentre observational study. Lancet Neurol. 2018; 17 (9): 782-789.
14. Willer B S, Haider M N, Wilber C, Esopenko C, Turner M, Leddy J. Long-Term Neurocognitive, Mental Health Consequences of Contact Sports. Clin Sports Med. 2021; 40 (1): 173-186.
15. N. Hotta, T. Tadokoro, J. Henry, D. Koga, K. Kawata, H. Ishida, Y. Oguma, A. Hirata, M. Mitsuhashi, K. Yoshitani, Monitoring of Post-Brain Injuries By Measuring Plasma Levels of Neuron-Derived Extracellular Vesicles. Biomark. Insights. 17 (2022) 11772719221128145.
16. K. Kawata, M. Mitsuhashi, R. Aldret, A preliminary report on brain-derived extracellular vesicle as novel blood biomarkers for sport-related concussions. Front. Neurol. 9 (2018) 239.
17. M. A. Edwardson, M. Mitsuhashi, D. V. Epps, Elevation of astrocyte-derived extracellular vesicles over the first month post-stroke in humans. Sci. Rep. 14 (2024) 5272.
18. R. de Rooij, E. Kuhl. Physical Biology of Axonal Damage. Front. Cell. Neurosci. 12 (2018) 144.
19. W. L. Maxwell, C. Watt, D. I. Graham, T. A. Gennarelli, Ultrastructural evidence of axonal shearing as a result of lateral acceleration of the head in non-human primates. Acta Neuropathol. 86 (1993) 136-44.
20. I. Lundgaard, A. Luzhynskaya, J. H. Stockley, Z. Wang, K. A. Evans, M. Swire, K. Volbracht, H. O. Gautier, R. J. Franklin, Charles Ffrench-Constant, D. Attwell, R. T. Karadottir. Neuregulin and BDNF induce a switch to NMDA receptor-dependent myelination by oligodendrocytes. PLOS Biol. 12 (2013) e1001743.
21. J. Langhnoja, L. Buch, P. Pillai. Potential role of NGF, BDNF, and their receptors in oligodendrocytes differentiation from neural stem cell: An in vitro study. Cell Biol. Int. 45 (2021) 432-446.
22. Q. Cao, Q. He, Y. Wang, X. Cheng, R. M. Howard, Y. Zhang, W. H. DeVries, C. B. Shields, D. S. Magnuson, X. M. Xu, D. H. Kim, S. R, Whittemore. Transplantation of ciliary neurotrophic factor-expressing adult oligodendrocyte precursor cells promotes remyelination and functional recovery after spinal cord injury. J. Neurosci. 30 (2010) 2989-3001.
23. M. Y. Cui, Y. Q. Fu, Z. L. Li, Y. Zheng, Y. Yu, C. Zhang, Y. Q. Zhang, B. R. Gao, W. Y. Chen, T. L. Lee, M. H. Won, M. Liao, Y. Jian, B. H. Chen, Neuregulin-1/PI3K signaling effects on oligodendrocyte proliferation, remyelination and behaviors deficit in a male mouse model of ischemic stroke. Exp. Neurol. 362 (2023) 114323.
24. R. M. Kalbfell, D. J. Rettke, K. Mackie, K. Ejima, J. Harezlak, I. L., Alexander, J. Wager-Miller, B. D. Johnson, S. D. Newman, K. Kawata. iScience. 26 (2023) 106948.
25. Z. W. Bevilacqua, M. E. Huibregtse, K. Kawata, In vivo protocol of controlled subconcussive head impacts for the validation of field study data. J. Vis. Exp. 146 (2019) e59381.
26. C. Dallner, A. G. Woods, T. Deller, M. Kirsch, H. D. Hofmann, CNTF and CNTF receptor alpha are constitutively expressed by astrocytes in the mouse brain. Glia. 37 (2002) 374-8.
27. J. Y. Baek, J. Y. Jeong, K. I. Kim, S-Y. Won, Y. C. Chung, J. H. Nam, E. J. Cho, T-B. Ahn, E. Bok, W-H. Shin, B. K. Jin, Inhibition of Microglia-Derived Oxidative Stress by Ciliary Neurotrophic Factor Protects Dopamine Neurons In Vivo from MPP+Neurotoxicity. Int. J. Mol. Sci. 19 (2018) 3543.
28. H. Ding, J. Chen, M. Su, Z. Lin, H. Zhan, F. Yang F, W. Li, J. Xie, Y. Huang, X. Liu, B. Liu, X. Zhou, BDNF promotes activation of astrocytes and microglia contributing to neuroinflammation in and mechanical allodynia cyclophosphamide-induced cystitis. J. Neuroinflammation. 17 (2020) 19.
29. Y. Xiang, T. Liu, H. Yang, F. Gao, H. Xiang, A. Manyande, Y. Tian, X. Tian, NRG1-ErbB signalling promotes microglia activation contributing to incision-induced mechanical allodynia. Eur. J. Pain. 19 (2015) 686-94.
30. Humble S S, Wilson L D, Wang L, et al. Prognosis of diffuse axonal injury with traumatic brain injury. J Trauma Acute Care Surg. 2018; 85 (1): 155-159.
31. Fiandaca M S, Kapogiannis D, Mapstone M, et al. Identification of preclinical Alzheimer's disease by a profile of pathogenic proteins in neurally derived blood exosomes: A case-control study. Alzheimers Dement. 2015; 11 (6): 600-7.
32. Delic V, Beck K D, Pang K C H, Citron B A. Biological Links Between Traumatic Brain Injury and Parkinson's Disease. Acta Neuropathologica Communications. 2020; 8:45.
33. Calabresi P, Di Lazzaro G, Marino G, Campanelli F, Ghiglieri V. Advances in Understanding the Function of Alpha-Synuclein: Implications for Parkinson's Disease. Brain 2023; 146:3587-3597.
34. Negi S, Khurana N, Duggal N. The Misfolding Mystery: A-Synuclein and the Pathogenesis of Parkinson's Disease. Neurochemistry International. 2024; 177:105760.
35. Freundt E C, Maynard N, Clancy E K, et al. Neuron-to-Neuron Transmission of A-Synuclein Fibrils Through Axonal Transport. Annals of Neurology. 2012; 72:517-524.
36. Gaudet C E, Weyandt L L. Immediate Post-Concussion and Cognitive Testing (ImPACT): a systematic review of the prevalence and assessment of invalid performance. Clin Neuropsychol. 2017; 31 (1): 43-58.
37. Khokhar B, Jorgensen-Wagers K, Marion D, Kiser S. Military Acute Concussion Evaluation: A Report on Clinical Usability, Utility, and User's Perceived Confidence. J Neurotrauma. 2021; 38 (2): 210-217.
38. Trbovich A M, Fazio-Sumrok V, Preszler J, et al. Discriminating Young Children with Concussion in an Outpatient Specialty Clinic from Healthy Controls Using the Child Sports Concussion Assessment Tool-5. J Pediatr. 2023; 257:113380.
39. Mangum L C, Skibski A, Devorski L, Slater L. Balance Error Scoring System Performance Differences in Figure Skaters Based on Discipline. Int J Sports Phys Ther. 2023; 18 (4): 898-904.
40. Brett B L, Breedlove K, McAllister T W, et al. Investigating the Range of Symptom Endorsement at Initiation of a Graduated Return-to-Play Protocol After Concussion and Duration of the Protocol: A Study From the National Collegiate Athletic Association-Department of Defense Concussion, Assessment, Research, and Education (CARE) Consortium. Am Sports Med. 2020; 48 (6): 1476-1484.
41. Herring S A, Margot P, Ben K W, et al. Team Physician Consensus Statement: Return to Sport/Return to Play and the Team Physician: A Team Physician Consensus Statement—2023 Update. Med Sci Sports Exerc. 2024; 56:767-775.
42. Wong A W, Xiao J, Kemper D, Kilpatrick T J, Murray SS. Oligodendroglial Expression of TrkB Independently Regulates Myelination and Progenitor Cell Proliferation. The Journal of Neuroscience. 2013; 33 (11): 4947-57.
43. Coleman M P, Hoke A. Programmed Axon Degeneration: From Mouse to Mechanism to Medicine. Nature Reviews. Neuroscience. 2020; 21 (4): 183-196.
44. Horwitz R I, Charlson M E, Singer B H. Medicine Based Evidence and Personalized Care of Patients. European Journal of Clinical Investigation. 2018; 48 (7): e12945.
45 Smith R, Capotosti F, Schain M, et al. The a-synuclein PET tracer [18F] ACI-12589 distinguishes multiple system atrophy from other neurodegenerative diseases. Nat Commun. 2023; 14 (1): 6750.
46. Loomis W F, Fuller D, Gutierrez E, Groisman A, Rappel W J. Innate Non-Specific Cell Substratum Adhesion. PloS One. 2012; e42033.

We claim:

1. A method of identifying at least one biomolecule from a patient, comprising:
collecting at least one biofluid from a patient,
isolating at least one exosome from the biofluid, and
identifying at least one biomolecule from the at least one exosome, wherein the at least one biomolecule is bound to the at least one exosome and is locally-released from the patient.

2. The method of claim 1, wherein the at least one biomolecule comprises a secretory protein, a neurotrophic factor, a growth factor, a cytokine, a chemokine, a pre-toxic molecule, a toxic molecule, or a combination thereof.

3. The method of claim 1, wherein the at least one biofluid is selected from blood, cerebrospinal fluid, urine, saliva, stool, luminal fluid, ascites, pleural effusion, or a combination thereof.

4. The method of claim 1, wherein the at least one biofluid comprises at least two independent exosomes, wherein the at least two independent exosomes are different from one another.

5. The method of claim 1, wherein the at least one exosome is neuron-derived, astrocyte-derived, oligodendrocyte-derived, tumor-derived, microglia-derived, or a combination thereof.

6. The method of claim 5, wherein the at least one oligodendrocyte-derived exosome is isolated by anti-MOG.

7. The method of claim 1, wherein the at least one biomolecule is used to identify asymptomatic brain injury or trauma in the patient.

8. The method of claim 1, wherein the at least one biomolecule is used to identify a severity of asymptomatic brain injury or trauma in the patient.

9. The method of claim 1, wherein the at least one biomolecule is used to identify the success of treatment of asymptomatic brain injury or trauma in the patient as compared with an initial level of the at least one biomolecule that was used to identify a severity of asymptomatic brain injury or trauma in the patient.

10. An assay, wherein the assay comprises the steps of:
obtaining a biological sample comprising at least one vesicle from a patient,
enriching the at least one vesicle, such that the at least one vesicle expresses a first biomarker and a second biomarker.

11. The assay of claim 10, wherein the assay is used to identify asymptomatic brain injury or trauma in the patient.

12. The assay of claim 10, wherein the at least one vesicle comprises an extracellular vesicle that is derived from at least one oligodendrocyte.

13. The assay of claim 10, wherein the first biomarker comprises myelin oligodendrocyte glycoprotein.

14. The assay of claim 10, wherein the second biomarker comprises a neuron-specific protein (e.g., synaptosome associated protein 25 (SNAP25), neurogranin (NRGN), tau, phosphorylated tau, αβ-42, αβ-40, along with aggregated forms, and synaptophysin), an astrocyte-specific protein (e.g., glial fibrillary acidic protein (GFAP) and excitatory amino acid transporter 1 (EAAT1)), a microglia-specific protein (CD11b), an oligodendrocyte-specific protein (e.g., myelin basic protein (MBP), an oligodendrocyte myelin glycoprotein (OMG), a cytosolic protein (e.g., glyceraldehyde-3-phosphate dehydrogenase (GAPDH), alpha-synuclein (SNCA), cathepsin D (CTSD), AchE, LAMP1, REST, SYT, TH, SYP, SYNPO, PSD95, SV2A, GYS, HSP70, BACE, SYMPO, NEFL, caspase, ubiquitin, PSEN1, GSK, PLAP, CSH1, PSG1, or FasL), a chemokine (CX3CL1, CCLs, CXCLs) or cytokine (interleukins, such as IL1b, IL34, IL12B or FasL).

15. The assay of claim 10, further comprising:
measuring the level of the at least one vesicle in the biological sample, and
comparing the level of the at least one vesicle in the biological sample to a control level of the at least one vesicle in a control biological sample, wherein an increase in the at least one vesicle in the biological sample as compared to the control level of the at least one vesicle in a control biological sample is an indicator of asymptomatic brain injury or trauma in the patient.

16. The assay of claim 15, wherein the control biological sample comprises a biological sample from the same patient at a different point in time.
